SEQUENCE LISTING (12 sequences)

SEQ ID NO: 1 - protein, 379 aa, Homo sapiens [sequence omitted]

SEQ ID NO: 2 - DNA, 1140 bp, Homo sapiens
  source 1..1140: mol_type = genomic DNA
atgccccactccagcctgcatccatccatcccgtgtcccaggggtcacggggcccagaaggcagccttggttctgctgagtgcctgcctggtgaccctttgggggctaggagagccaccagagcacactctccggtacctggtcctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgggctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 3 - DNA, 1140 bp, unidentified
  source 1..1140: mol_type = unassigned DNA
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgggctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 4 - DNA, 1140 bp, unidentified
  source 1..1140: mol_type = unassigned DNA
atgccccactccagcctgcatccatccatcccgtgtcccaggggtcacggggcccagaaggcagccttggttctgctgagtgcctgcctggtgaccctttgggggctaggagagccaccagagcacactctccggtacctggttctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgggctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 5 - protein, 796 aa, Homo sapiens
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGSTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 6 - DNA, 6359 bp, Homo sapiens
  source 1..6359: mol_type = genomic DNA
ccagctcttacctggtagtgtccatcactaagtgtaaagtttggggagaatggagtgtggcagtccgtagaaggaaaactcagcatgctgatccactcatgtcagtaccattggggtaaaagggacactgcctagctactcattaagtactagttggaactcatcctcactgtttgaatcagagacagagagaggacaaatttctttctgcctttcatcagggagtctattttcctgagacaaagcttgagtgtggcaggtttgccctaacacagttccagttacatttttgtgagtgaagatgctcctggatcctgccattacatttctgtgcttctttgttttcagtggccaagtggatttttcaaatgtatgtgtgtctctgtgggtgttctgatgggcagttgggagaagttatatggatcagtgaagagcttttaaccatacgccattcagtaataaaagttccccaactcagagttttcactcttccaaagaaagaatttggactcatatcaagatgctctgaagaagaacaaccctttaggatagccactgcaacatcatgaccaaagacaaagaacctattgttaaaagcttccattttgtttgccttatgatcataatagttggaaccagaatccagttctccgacggaaatgaatttgcagtagacaagtcaaaaagaggtcttattcatgttccaaaagacctaccgctgaaaaccaaagtcttagatatgtctcagaactacatcgctgagcttcaggtctctgacatgagctttctatcagagttgacagttttgagactttcccataacagaatccagctacttgatttaagtgttttcaagttcaaccaggatttagaatatttggatttatctcataatcagttgcaaaagatatcctgccatcctattgtgagtttcaggcatttagatctctcattcaatgatttcaaggccctgcccatctgtaaggaatttggcaacttatcacaactgaatttcttgggattgagtgctatgaagctgcaaaaattagatttgctgccaattgctcacttgcatctaagttatatccttctggatttaagaaattattatataaaagaaaatgagacagaaagtctacaaattctgaatgcaaaaacccttcaccttgtttttcacccaactagtttattcgctatccaagtgaacatatcagttaatactttagggtgcttacaactgactaatattaaattgaatgatgacaactgtcaagttttcattaaatttttatcagaactcaccagaggttcaaccttactgaattttaccctcaaccacatagaaacgacttggaaatgcctggtcagagtctttcaatttctttggcccaaacctgtggaatatctcaatatttacaatttaacaataattgaaagcattcgtgaagaagattttacttattctaaaacgacattgaaagcattgacaatagaacatatcacgaaccaagtttttctgttttcacagacagctttgtacaccgtgttttctgagatgaacattatgatgttaaccatttcagatacaccttttatacacatgctgtgtcctcatgcaccaagcacattcaagtttttgaactttacccagaacgttttcacagatagtatttttgaaaaatgttccacgttagttaaattggagacacttatcttacaaaagaatggattaaaagaccttttcaaagtaggtctcatgacgaaggatatgccttctttggaaatactggatgttagctggaattctttggaatctggtagacataaagaaaactgcacttgggttgagagtatagtggtgttaaatttgtcttcaaatatgcttactgactctgttttcagatgtttacctcccaggatcaaggtacttgatcttcacagcaataaaataaagagcgttcctaaacaagtcgtaaaactggaagctttgcaagaactcaatgttgctttcaattctttaactgaccttcctggatgtggcagctttagcagcctttctgtattgatcattgatcacaattcagtttcccacccatcggctgatttcttccagagctgccagaagatgaggtcaataaaagcaggggacaatccattccaatgtacctgtgagctaagagaatttgtcaaaaatatagaccaagtatcaagtgaagtgttagagggctggcctgattcttataagtgtgactacccagaaagttatagaggaagcccactaaaggactttcacatgtctgaattatcctgcaacataactctgctgatcgtcaccatcggtgccaccatgctggtgttggctgtgactgtgacctccctctgcatctacttggatctgccctggtatctcaggatggtgtgccagtggacccagactcggcgcagggccaggaacatacccttagaagaactccaaagaaacctccagtttcatgcttttatttcatatagtgaacatgattctgcctgggtgaaaagtgaattggtaccttacctagaaaaagaagatatacagatttgtcttcatgagagaaactttgtccctggcaagagcattgtggaaaatatcatcaactgcattgagaagagttacaagtccatctttgttttgtctcccaactttgtccagagtgagtggtgccattacgaactctattttgcccatcacaatctctttcatgaaggatctaataacttaatcctcatcttactggaacccattccacagaacagcattcccaacaagtaccacaagctgaaggctctcatgacgcagcggacttatttgcagtggcccaaggagaaaagcaaacgtgggctcttttgggctaacattagagccgcttttaatatgaaattaacactagtcactgaaaacaatgatgtgaaatcttaaaaaaatttaggaaattcaacttaagaaaccattatttacttggatgatggtgaatagtacagtcgtaagtaactgtctggaggtgcctccattatcctcatgccttcaggaaagacttaacaaaaacaatgtttcatctggggaactgagctaggcggtgaggttagcctgccagttagagacagcccagtctcttctggtttaatcattatgtttcaaattgaaacagtctcttttgagtaaatgctcagtttttcagctcctctccactctgctttcccaaatggattctgttgtgagcaagagtttatatggcttcatggcagcaagggaacagtcaacttcagcatcatatgcaccagtcctcggagtgccctgtgaatcatattggtctttgggtcagtgtcatcattctcttcaagtctggggcttggggaaaaaattagatcagctacggcatataaaaaagtcttttgtttcacatatgtgtaatagcttatttaattttttatcctgctacacaaatatgtaattaaccaatgaggactcatgacttgatagtgtatgtatgtaaagggatatatggacttaatcataagctgttgaggtgaaagacgtggatccacctgctttccaagaaaactcggccaaatttatttgcagctggatattgaatgggacttttctggttgtcttagaattctggctaaaggctcaaagctgacgaaagacagtaactgcaccaacatgatactagacacagccagtctggacttatcaaaagagcagaaagagaccaatgactcccagtccgtattatccatctctagaagactagagtcaaaagcgtgattaaagagtcattaagcggaggttctaggccatagggagattgctttgaatttcttgcagacaagtgtgagggactcagcatggtagaaggtagcctggcatcccactccaagactgaaagcttgcagagtaacaggagcacacaggttcagtgcagcagatgtggtgtggcttgagaattcttggaagagcttgatgagtgtttgctggagtccgagggtgggcactgggaacacagagactggtaaatagtgtttggcaaatacaagtgcttgatgaatatttgttgaatgaatagatgagttcttcccccctggggaattcaggaggtgaaaggttggcttgagcacccaaaatggcaggatgagagaagagaagcactgatagcaacctgccctcccattattgacatggtaaaaggatgtgaatttcttcacatggctttgactatggaagagtagctgggcttgcattgtcatgacgggatatcagccaacagggtagcctgttgtgcaaagaaactatagcagtaagaggacacggggttaggcagaagaggggtttggggtggaggttgctgcaagaggtcagccagataatgtggccctgcatcatggaactgtgcaatgtggggtacactcaaggccctccaataactcacagatgtgccctatgaaaaagccagcatttggactctgccatagcagctggcaggatcatgctggcctgtctgccttattcaatagttaactacaggaagatctgctcctctttgtgtaataccctcttcccttgcaatggcatagggacatctagaatatagagaagacagagacaatggaggaagagtaaagaaactgactatatgccttcgtcatttcactgcaaggaaggccaagcagatttttgaatgaggtgtgagattgctgttaaattggactggcctggacattttaatcccttaaatagaggtgcaatgactaaagtgagatttgtcactaaaatttatggtatctgcccaagattcaggagtgatgatgggaggagatccaacagaactttgttgtaaggcaatggttagagaaaaatgaagccctcgctttctggacttagttcattcaataaaccagtttcggccaggcacgttggctcacatctataatcccagtactgtgggaggctgaggcaggtggatcacttgaggtcaggagttcgagaccagcctggccaacatggtgaaaccctgtctgtactaaaaatacaaaaattagccgggtgtggtggtgtgcacctgtagtcccagctactcgggaggctgaggcaggaaaatcacttgaacctgggagacagaggctgtagtgagctgagacagcgctactgtactccccgctgggcaacagagtgagactccatctcaaaaaagttaaaagaaaaaaaatctggtttcataatagctgtaacgaaataagccttaatgatattttattagcatcatcttctgtctgcattagcccttccttgctcttcaggagaacaacatttgttttcctccctaggctctatcccaaacggcacattcttccacaacccctgttgaacagattttttaaactgttgcctaatctaaaaacaataaaaacaacaaacaaccacagtaacaacaacgacaaaaaaaactgccacagattctaaataatcagatctttttaaatggtatcaatgtttcccacaaaatattgttgacattgaaaatatagaattttagcattaattttgttaaacctacatcccctcggcagaggggcctccctgcatcccagtggaaagtaggttcctcacagtcctctccgtcacattcttcccatttcttttcttcacagaacacatcactgtctaaaattatcttgtttgcttagttgcttactcatcttcttcttctctcctctgaagtctaagctccaggaaaaagggagacttctccacctgttccctgcctctccccagtgccgaggggacactgtgcaccccattgtagatgcgcagtaaaaactcgtgggatgagcaaatgactctgaaacggtcccatgcgggaaatgtccatgaagtcctggattttatctaaaaagcccaggcaggggggggcgggggcggcggggctacagttccacgctgagctgcctcctggccgctcgtccccgccgcagtgcctgggcggcccgggcgcccgaccttggccgtggacaccttcgcggtgggtgctgctcctccccatctgccactggaagatgctggggcgacccggctccaggtttagcaggacactgagaaaagggaatggctgcctttcggaggctgggtgagcccttctctgtgcctcacctgcccgccccacagcggccctgcacctcgtcccacggggcccattgccccggtaggatgcgcgcttttgttttgagggtcaggcatcttccctgccgtcgtttctgggaggttgaaaaattgatccagaaagacctaaaacaaaaaacaaagaaaaacaaaaaagcgaaaa

SEQ ID NO: 7 - protein, 796 aa, unidentified
MTKDKEPIVKSFHFVCLMIIIVGTRIQFSDGNEFAVDKSKRGLIHVPKDLPLKTKVLDMSQNYIAELQVSDMSFLSELTVLRLSHNRIQLLDLSVFKFNQDLEYLDLSHNQLQKISCHPIVSFRHLDLSFNDFKALPICKEFGNLSQLNFLGLSAMKLQKLDLLPIAHLHLSYILLDLRNYYIKENETESLQILNAKTLHLVFHPTSLFAIQVNISVNTLGCLQLTNIKLNDDNCQVFIKFLSELTRGPTLLNFTLNHIETTWKCLVRVFQFLWPKPVEYLNIYNLTIIESIREEDFTYSKTTLKALTIEHITNQVFLFSQTALYTVFSEMNIMMLTISDTPFIHMLCPHAPSTFKFLNFTQNVFTDSIFEKCSTLVKLETLILQKNGLKDLFKVGLMTKDMPSLEILDVSWNSLESGRHKENCTWVESIVVLNLSSNMLTDSVFRCLPPRIKVLDLHSNKIKSVPKQVVKLEALQELNVAFNSLTDLPGCGSFSSLSVLIIDHNSVSHPSADFFQSCQKMRSIKAGDNPFQCTCELREFVKNIDQVSSEVLEGWPDSYKCDYPESYRGSPLKDFHMSELSCNITLLIVTIGATMLVLAVTVTSLCIYLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 8 - DNA, 6359 bp, unidentified
  source 1..6359: mol_type = unassigned DNA
ccagctcttacctggtagtgtccatcactaagtgtaaagtttggggagaatggagtgtggcagtccgtagaaggaaaactcagcatgctgatccactcatgtcagtaccattggggtaaaagggacactgcctagctactcattaagtactagttggaactcatcctcactgtttgaatcagagacagagagaggacaaatttctttctgcctttcatcagggagtctattttcctgagacaaagcttgagtgtggcaggtttgccctaacacagttccagttacatttttgtgagtgaagatgctcctggatcctgccattacatttctgtgcttctttgttttcagtggccaagtggatttttcaaatgtatgtgtgtctctgtgggtgttctgatgggcagttgggagaagttatatggatcagtgaagagcttttaaccatacgccattcagtaataaaagttccccaactcagagttttcactcttccaaagaaagaatttggactcatatcaagatgctctgaagaagaacaaccctttaggatagccactgcaacatcatgaccaaagacaaagaacctattgttaaaagcttccattttgtttgccttatgatcataatagttggaaccagaatccagttctccgacggaaatgaatttgcagtagacaagtcaaaaagaggtcttattcatgttccaaaagacctaccgctgaaaaccaaagtcttagatatgtctcagaactacatcgctgagcttcaggtctctgacatgagctttctatcagagttgacagttttgagactttcccataacagaatccagctacttgatttaagtgttttcaagttcaaccaggatttagaatatttggatttatctcataatcagttgcaaaagatatcctgccatcctattgtgagtttcaggcatttagatctctcattcaatgatttcaaggccctgcccatctgtaaggaatttggcaacttatcacaactgaatttcttgggattgagtgctatgaagctgcaaaaattagatttgctgccaattgctcacttgcatctaagttatatccttctggatttaagaaattattatataaaagaaaatgagacagaaagtctacaaattctgaatgcaaaaacccttcaccttgtttttcacccaactagtttattcgctatccaagtgaacatatcagttaatactttagggtgcttacaactgactaatattaaattgaatgatgacaactgtcaagttttcattaaatttttatcagaactcaccagaggtccaaccttactgaattttaccctcaaccacatagaaacgacttggaaatgcctggtcagagtctttcaatttctttggcccaaacctgtggaatatctcaatatttacaatttaacaataattgaaagcattcgtgaagaagattttacttattctaaaacgacattgaaagcattgacaatagaacatatcacgaaccaagtttttctgttttcacagacagctttgtacaccgtgttttctgagatgaacattatgatgttaaccatttcagatacaccttttatacacatgctgtgtcctcatgcaccaagcacattcaagtttttgaactttacccagaacgttttcacagatagtatttttgaaaaatgttccacgttagttaaattggagacacttatcttacaaaagaatggattaaaagaccttttcaaagtaggtctcatgacgaaggatatgccttctttggaaatactggatgttagctggaattctttggaatctggtagacataaagaaaactgcacttgggttgagagtatagtggtgttaaatttgtcttcaaatatgcttactgactctgttttcagatgtttacctcccaggatcaaggtacttgatcttcacagcaataaaataaagagcgttcctaaacaagtcgtaaaactggaagctttgcaagaactcaatgttgctttcaattctttaactgaccttcctggatgtggcagctttagcagcctttctgtattgatcattgatcacaattcagtttcccacccatcggctgatttcttccagagctgccagaagatgaggtcaataaaagcaggggacaatccattccaatgtacctgtgagctaagagaatttgtcaaaaatatagaccaagtatcaagtgaagtgttagagggctggcctgattcttataagtgtgactacccagaaagttatagaggaagcccactaaaggactttcacatgtctgaattatcctgcaacataactctgctgatcgtcaccatcggtgccaccatgctggtgttggctgtgactgtgacctccctctgcatctacttggatctgccctggtatctcaggatggtgtgccagtggacccagactcggcgcagggccaggaacatacccttagaagaactccaaagaaacctccagtttcatgcttttatttcatatagtgaacatgattctgcctgggtgaaaagtgaattggtaccttacctagaaaaagaagatatacagatttgtcttcatgagagaaactttgtccctggcaagagcattgtggaaaatatcatcaactgcattgagaagagttacaagtccatctttgttttgtctcccaactttgtccagagtgagtggtgccattacgaactctattttgcccatcacaatctctttcatgaaggatctaataacttaatcctcatcttactggaacccattccacagaacagcattcccaacaagtaccacaagctgaaggctctcatgacgcagcggacttatttgcagtggcccaaggagaaaagcaaacgtgggctcttttgggctaacattagagccgcttttaatatgaaattaacactagtcactgaaaacaatgatgtgaaatcttaaaaaaatttaggaaattcaacttaagaaaccattatttacttggatgatggtgaatagtacagtcgtaagtaactgtctggaggtgcctccattatcctcatgccttcaggaaagacttaacaaaaacaatgtttcatctggggaactgagctaggcggtgaggttagcctgccagttagagacagcccagtctcttctggtttaatcattatgtttcaaattgaaacagtctcttttgagtaaatgctcagtttttcagctcctctccactctgctttcccaaatggattctgttgtgagcaagagtttatatggcttcatggcagcaagggaacagtcaacttcagcatcatatgcaccagtcctcggagtgccctgtgaatcatattggtctttgggtcagtgtcatcattctcttcaagtctggggcttggggaaaaaattagatcagctacggcatataaaaaagtcttttgtttcacatatgtgtaatagcttatttaattttttatcctgctacacaaatatgtaattaaccaatgaggactcatgacttgatagtgtatgtatgtaaagggatatatggacttaatcataagctgttgaggtgaaagacgtggatccacctgctttccaagaaaactcggccaaatttatttgcagctggatattgaatgggacttttctggttgtcttagaattctggctaaaggctcaaagctgacgaaagacagtaactgcaccaacatgatactagacacagccagtctggacttatcaaaagagcagaaagagaccaatgactcccagtccgtattatccatctctagaagactagagtcaaaagcgtgattaaagagtcattaagcggaggttctaggccatagggagattgctttgaatttcttgcagacaagtgtgagggactcagcatggtagaaggtagcctggcatcccactccaagactgaaagcttgcagagtaacaggagcacacaggttcagtgcagcagatgtggtgtggcttgagaattcttggaagagcttgatgagtgtttgctggagtccgagggtgggcactgggaacacagagactggtaaatagtgtttggcaaatacaagtgcttgatgaatatttgttgaatgaatagatgagttcttcccccctggggaattcaggaggtgaaaggttggcttgagcacccaaaatggcaggatgagagaagagaagcactgatagcaacctgccctcccattattgacatggtaaaaggatgtgaatttcttcacatggctttgactatggaagagtagctgggcttgcattgtcatgacgggatatcagccaacagggtagcctgttgtgcaaagaaactatagcagtaagaggacacggggttaggcagaagaggggtttggggtggaggttgctgcaagaggtcagccagataatgtggccctgcatcatggaactgtgcaatgtggggtacactcaaggccctccaataactcacagatgtgccctatgaaaaagccagcatttggactctgccatagcagctggcaggatcatgctggcctgtctgccttattcaatagttaactacaggaagatctgctcctctttgtgtaataccctcttcccttgcaatggcatagggacatctagaatatagagaagacagagacaatggaggaagagtaaagaaactgactatatgccttcgtcatttcactgcaaggaaggccaagcagatttttgaatgaggtgtgagattgctgttaaattggactggcctggacattttaatcccttaaatagaggtgcaatgactaaagtgagatttgtcactaaaatttatggtatctgcccaagattcaggagtgatgatgggaggagatccaacagaactttgttgtaaggcaatggttagagaaaaatgaagccctcgctttctggacttagttcattcaataaaccagtttcggccaggcacgttggctcacatctataatcccagtactgtgggaggctgaggcaggtggatcacttgaggtcaggagttcgagaccagcctggccaacatggtgaaaccctgtctgtactaaaaatacaaaaattagccgggtgtggtggtgtgcacctgtagtcccagctactcgggaggctgaggcaggaaaatcacttgaacctgggagacagaggctgtagtgagctgagacagcgctactgtactccccgctgggcaacagagtgagactccatctcaaaaaagttaaaagaaaaaaaatctggtttcataatagctgtaacgaaataagccttaatgatattttattagcatcatcttctgtctgcattagcccttccttgctcttcaggagaacaacatttgttttcctccctaggctctatcccaaacggcacattcttccacaacccctgttgaacagattttttaaactgttgcctaatctaaaaacaataaaaacaacaaacaaccacagtaacaacaacgacaaaaaaaactgccacagattctaaataatcagatctttttaaatggtatcaatgtttcccacaaaatattgttgacattgaaaatatagaattttagcattaattttgttaaacctacatcccctcggcagaggggcctccctgcatcccagtggaaagtaggttcctcacagtcctctccgtcacattcttcccatttcttttcttcacagaacacatcactgtctaaaattatcttgtttgcttagttgcttactcatcttcttcttctctcctctgaagtctaagctccaggaaaaagggagacttctccacctgttccctgcctctccccagtgccgaggggacactgtgcaccccattgtagatgcgcagtaaaaactcgtgggatgagcaaatgactctgaaacggtcccatgcgggaaatgtccatgaagtcctggattttatctaaaaagcccaggcaggggggggcgggggcggcggggctacagttccacgctgagctgcctcctggccgctcgtccccgccgcagtgcctgggcggcccgggcgcccgaccttggccgtggacaccttcgcggtgggtgctgctcctccccatctgccactggaagatgctggggcgacccggctccaggtttagcaggacactgagaaaagggaatggctgcctttcggaggctgggtgagcccttctctgtgcctcacctgcccgccccacagcggccctgcacctcgtcccacggggcccattgccccggtaggatgcgcgcttttgttttgagggtcaggcatcttccctgccgtcgtttctgggaggttgaaaaattgatccagaaagacctaaaacaaaaaacaaagaaaaacaaaaaagcgaaaa

SEQ ID NO: 9 - protein, 811 aa, Homo sapiens
MRLIRNIYIFCSIVMTAEGDAPELPEERELMTNCSNMSLRKVPADLTPATTTLDLSYNLLFQLQSSDFHSVSKLRVLILCHNRIQQLDLKTFEFNKELRYLDLSNNRLKSVTWYLLAGLRYLDLSFNDFDTMPICEEAGNMSHLEILGLSGAKIQKSDFQKIAHLHLNTVFLGFRTLPHYEEGSLPILNTTKLHIVLPMDTNFWVLLRDGIKTSKILEMTNIDGKSQFVSYEMQRNLSLENAKTSVLLLNKVDLLWDDLFLILQFVWHTSVEHFQIRNVTFGGKAYLDHNSFDYSNTVMRTIKLEHVHFRVFYIQQDKIYLLLTKMDIENLTISNAQMPHMLFPNYPTKFQYLNFANNILTDELFKRTIQLPHLKTLILNGNKLETLSLVSCFANNTPLEHLDLSQNLLQHKNDENCSWPETVVNMNLSYNKLSDSVFRCLPKSIQILDLNNNQIQTVPKETIHLMALRELNIAFNFLTDLPGCSHFSRLSVLNIEMNFILSPSLDFVQSCQEVKTLNAGRNPFRCTCELKNFIQLETYSEVMMVGWSDSYTCEYPLNLRGTRLKDVHLHELSCNTALLIVTIVVIMLVLGLAVAFCCLHFDLPWYLRMLGQCTQTWHRVRKTTQEQLKRNVRFHAFISYSEHDSLWVKNELIPNLEKEDGSILICLYESYFDPGKSISENIVSFIEKSYKSIFVLSPNFVQNEWCHYEFYFAHHNLFHENSDHIILILLEPIPFYCIPTRYHKLKALLEKKAYLEWPKDRRKCGLFWANLRAAINVNVLATREMYELQTFTELNEESRGSTISLMRTDCL

SEQ ID NO: 10 - DNA, 3991 bp, Homo sapiens
  source 1..3991: mol_type = genomic DNA
agccaattctgaccgtgtcaacgaatcatccacgcacctgcagctctgctgagagagtgcaagccgtgggaattcagcagctgaatatcaagacctttgaattcaacaagaagttaagacatttatagttgtctaacaacagactgaagattgtggcttggtattcactggcaggtttcagacatttagatctttcttttaatgactaacaccatgcctatctgtggagaagctggcaacatgtcacacctggaaattgtttttcaacattaatactattatttggcagtaatccagattgcttttgccaccaacctgaagacatatagaggcagaaggacaggaataattctatttgtttcctgttttgaaacttccatctgtaaggctatcaaaaggagatgtgagagagggtattgagtctggcctgacaatgcagttcttaaaccaaaggtccattatgcttctcctctctgagaatcctgacttacctcaacaacggagacatggcacagtagccagcttggagacttctcagccaatgctctgagatcaagtcgaagacccaatatacagggttttgagctcatcttcatcattcatatgaggaaataagtggtaaaatccttggaaatacaatgagactcatcagaaacatttacatattttgtagtattgttatgacagcagagggtgatgctccagagctgccagaagaaagggaactgatgaccaactgctccaacatgtctctaagaaaggttcccgcagacttgaccccagccacaacgacactggatttatcctataacctcctttttcaactccagagttcagattttcattctgtctccaaactgagagttttgattctatgccataacagaattcaacagctggatctcaaaacctttgaattcaacaaggagttaagatatttagatttgtctaataacagactgaagagtgtaacttggtatttactggcaggtctcaggtatttagatctttcttttaatgactttgacaccatgcctatctgtgaggaagctggcaacatgtcacacctggaaatcctaggtttgagtggggcaaaaatacaaaaatcagatttccagaaaattgctcatctgcatctaaatactgtcttcttaggattcagaactcttcctcattatgaagaaggtagcctgcccatcttaaacacaacaaaactgcacattgttttaccaatggacacaaatttctgggttcttttgcgtgatggaatcaagacttcaaaaatattagaaatgacaaatatagatggcaaaagccaatttgtaagttatgaaatgcaacgaaatcttagtttagaaaatgctaagacatcggttctattgcttaataaagttgatttactctgggacgaccttttccttatcttacaatttgtttggcatacatcagtggaacactttcagatccgaaatgtgacttttggtggtaaggcttatcttgaccacaattcatttgactactcaaatactgtaatgagaactataaaattggagcatgtacatttcagagtgttttacattcaacaggataaaatctatttgcttttgaccaaaatggacatagaaaacctgacaatatcaaatgcacaaatgccacacatgcttttcccgaattatcctacgaaattccaatatttaaattttgccaataatatcttaacagacgagttgtttaaaagaactatccaactgcctcacttgaaaactctcattttgaatggcaataaactggagacactttctttagtaagttgctttgctaacaacacacccttggaacacttggatctgagtcaaaatctattacaacataaaaatgatgaaaattgctcatggccagaaactgtggtcaatatgaatctgtcatacaataaattgtctgattctgtcttcaggtgcttgcccaaaagtattcaaatacttgacctaaataataaccaaatccaaactgtacctaaagagactattcatctgatggccttacgagaactaaatattgcatttaattttctaactgatctccctggatgcagtcatttcagtagactttcagttctgaacattgaaatgaacttcattctcagcccatctctggattttgttcagagctgccaggaagttaaaactctaaatgcgggaagaaatccattccggtgtacctgtgaattaaaaaatttcattcagcttgaaacatattcagaggtcatgatggttggatggtcagattcatacacctgtgaataccctttaaacctaaggggaactaggttaaaagacgttcatctccacgaattatcttgcaacacagctctgttgattgtcaccattgtggttattatgctagttctggggttggctgtggccttctgctgtctccactttgatctgccctggtatctcaggatgctaggtcaatgcacacaaacatggcacagggttaggaaaacaacccaagaacaactcaagagaaatgtccgattccacgcatttatttcatacagtgaacatgattctctgtgggtgaagaatgaattgatccccaatctagagaaggaagatggttctatcttgatttgcctttatgaaagctactttgaccctggcaaaagcattagtgaaaatattgtaagcttcattgagaaaagctataagtccatctttgttttgtctcccaactttgtccagaatgagtggtgccattatgaattctactttgcccaccacaatctcttccatgaaaattctgatcatataattcttatcttactggaacccattccattctattgcattcccaccaggtatcataaactgaaagctctcctggaaaaaaaagcatacttggaatggcccaaggataggcgtaaatgtgggcttttctgggcaaaccttcgagctgctattaatgttaatgtattagccaccagagaaatgtatgaactgcagacattcacagagttaaatgaagagtctcgaggttctacaatctctctgatgagaacagattgtctataaaatcccacagtccttgggaagttggggaccacatacactgttgggatgtacattgatacaacctttatgatggcaatttgacaatatttattaaaataaaaaatggttattcccttcatatcagtttctagaaggatttctaagaatgtatcctatagaaacaccttcacaagtttataagggcttatggaaaaaggtgttcatcccaggattgtttataatcatgaaaaatgtggccaggtgcagtggctcactcttgtaatcccagcactatgggaggccaaggtgggtgacccacgaggtcaagagatggagaccatcctggccaacatggtgaaaccctgtctctactaaaaatacaaaaattagctgggcgtgatggtgcacgcctgtagtcccagctacttgggaggctgaggcaggagaatcgcttgaacccgggaggtggcagttgcagtgagctgagatcgagccactgcactccagcctggtgacagagcgagactccatctcaaaaaaaagaaaaaaaaaaaagaaaaaaatggaaaacatcctcatggccacaaaataaggtctaattcaataaattatagtacattaatgtaatataatattacatgccactaaaaagaataaggtagctgtatatttcctggtatggaaaaaacatattaatatgttataaactattaggttggtgcaaaactaattgtggtttttgccattgaaatggcattgaaataaaagtgtaaagaaatctataccagatgtagtaacagtggtttgggtctgggaggttggattacagggagcatttgatttctatgttgtgtatttctataatgtttgaattgtttagaatgaatctgtatttcttttataagtagaaaaaaaataaagatagtttttacagccta

SEQ ID NO: 11 - protein, 811 aa, unidentified [sequence omitted]

SEQ ID NO: 12 - DNA, 3991 bp, unidentified
  source 1..3991: mol_type = unassigned DNA
agccaattctgaccgtgtcaacgaatcatccacgcacctgcagctctgctgagagagtgcaagccgtgggaattcagcagctgaatatcaagacctttgaattcaacaagaagttaagacatttatagttgtctaacaacagactgaagattgtggcttggtattcactggcaggtttcagacatttagatctttcttttaatgactaacaccatgcctatctgtggagaagctggcaacatgtcacacctggaaattgtttttcaacattaatactattatttggcagtaatccagattgcttttgccaccaacctgaagacatatagaggcagaaggacaggaataattctatttgtttcctgttttgaaacttccatctgtaaggctatcaaaaggagatgtgagagagggtattgagtctggcctgacaatgcagttcttaaaccaaaggtccattatgcttctcctctctgagaatcctgacttacctcaacaacggagacatggcacagtagccagcttggagacttctcagccaatgctctgagatcaagtcgaagacccaatatacagggttttgagctcatcttcatcattcatatgaggaaataagtggtaaaatccttggaaatacaatgagactcatcagaaacatttacatattttgtagtattgttatgacagcagagggtgatgctccagagctgccagaagaaagggaactgatgaccaactgctccaacatgtctctaagaaaggttcccgcagacttgaccccagccacaacgacactggatttatcctataacctcctttttcaactccagagttcagattttcattctgtctccaaactgagagttttgattctatgccataacagaattcaacagctggatctcaaaacctttgaattcaacaaggagttaagatatttagatttgtctaataacagactgaagagtgtaacttggtatttactggcaggtctcaggtatttagatctttcttttaatgactttgacaccatgcctatctgtgaggaagctggcaacatgtcacacctggaaatcctaggtttgagtggggcaaaaatacaaaaatcagatttccagaaaattgctcatctgcatctaaatactgtcttcttaggattcagaactcttcctcattatgaagaaggtagcctgcccatcttaaacacaacaaaactgcacattgttttaccaatggacacaaatttctgggttcttttgcgtgatggaatcaagacttcaaaaatattagaaatgacaaatatagatggcaaaagccaatttgtaagttatgaaatgcaacgaaatcttagtttagaaaatgctaagacatcggttctattgcttaataaagttgatttactctgggacgaccttttccttatcttacaatttgtttggcatacatcagtggaacactttcagatccgaaatgtgacttttggtggtaaggcttatcttgaccacaattcatttgactactcaaatactgtaatgagaactataaaattggagcatgtacatttcagagtgttttacattcaacaggataaaatctatttgcttttgaccaaaatggacatagaaaacctgacaatatcaaatgcacaaatgccacacatgcttttcccgaattatcctacgaaattccaatatttaaattttgccaataatatcttaacagacgagttgtttaaaagaactatccaactgcctcacttgaaaactctcattttgaatggcaataaactggagacactttctttagtaagttgctttgctaacaacacacccttggaacacttggatctgagtcaaaatctattacaacataaaaatgatgaaaattgctcatggccagaaactgtggtcaatatgaatctgtcatacaataaattgtctgattctgtcttcaggtgcttgcccaaaagtattcaaatacttgacctaaataataaccaaatccaaactgtacctaaagagactattcatctgatggccttacgagaactaaatattgcatttaattttctaactgatctccctggatgcagtcatttcagtagactttcagttctgaacattgaaatgaacttcattctcagcccatctctggattttgttcagagctgccaggaagttaaaactctaaatgcgggaagaaatccattccggtgtacctgtgaattaaaaaatttcattcagcttgaaacatattcagaggtcatgatggttggatggtcagattcatacacctgtgaataccctttaaacctaaggggaactaggttaaaagacgttcatctccacgaattatcttgcaacacagctctgttgattgtcaccattgtggttattatgctagttctggggttggctgtggccttctgctgtctccactttgatctgccctggtatctcaggatgctaggtcaatgcacacaaacatggcacagggttaggaaaacaacccaagaacaactcaagagaaatgtccgattccacgcatttatttcatacagtgaacatgattctctgtgggtgaagaatgaattgatccccaatctagagaaggaagatggttctatcttgatttgcctttatgaaagctactttgaccctggcaaaagcattagtgaaaatattgtaagcttcattgagaaaagctataagtccatctttgttttgtctcccaactttgtccagaatgagtggtgccattatgaattctactttgcccaccacaatctcttccatgaaaattctgatcatataattcttatcttactggaacccattccattctattgcattcccaccaggtatcataaactgaaagctctcctggaaaaaaaagcatacttggaatggcccaaggataggcgtaaatgtgggcttttctgggcaaaccttcgagctgctgttaatgttaatgtattagccaccagagaaatgtatgaactgcagacattcacagagttaaatgaagagtctcgaggttctacaatctctctgatgagaacagattgtctataaaatcccacagtccttgggaagttggggaccacatacactgttgggatgtacattgatacaacctttatgatggcaatttgacaatatttattaaaataaaaaatggttattcccttcatatcagtttctagaaggatttctaagaatgtatcctatagaaacaccttcacaagtttataagggcttatggaaaaaggtgttcatcccaggattgtttataatcatgaaaaatgtggccaggtgcagtggctcactcttgtaatcccagcactatgggaggccaaggtgggtgacccacgaggtcaagagatggagaccatcctggccaacatggtgaaaccctgtctctactaaaaatacaaaaattagctgggcgtgatggtgcacgcctgtagtcccagctacttgggaggctgaggcaggagaatcgcttgaacccgggaggtggcagttgcagtgagctgagatcgagccactgcactccagcctggtgacagagcgagactccatctcaaaaaaaagaaaaaaaaaaaagaaaaaaatggaaaacatcctcatggccacaaaataaggtctaattcaataaattatagtacattaatgtaatataatattacatgccactaaaaagaataaggtagctgtatatttcctggtatggaaaaaacatattaatatgttataaactattaggttggtgcaaaactaattgtggtttttgccattgaaatggcattgaaataaaagtgtaaagaaatctataccagatgtagtaacagtggtttgggtctgggaggttggattacagggagcatttgatttctatgttgtgtatttctataatgtttgaattgtttagaatgaatctgtatttcttttataagtagaaaaaaaataaagatagtttttacagccta

(19) United States
(12) Patent Application Publication
(10) Pub. No.: US 2024/0384356 A1
(43) Pub. Date: Nov. 21, 2024

(54) BIOMARKERS FOR STING AGONISTS AND METHODS OF USING THE SAME

(71) Applicant: EISAI R&D MANAGEMENT CO., LTD., Tokyo (JP)

(72) Inventors: Tenghui CHEN, Cambridge, MA (US); Antonio GUALBERTO, Cambridge, MA (US); Catherine SCHOLZ, Cambridge, MA (US)

(21) Appl. No.: 18/690,679

(22) PCT Filed: Sep. 2, 2022
(86) PCT No.: PCT/US2022/075948

§ 371 Date: Mar. 8, 2024

Related U.S. Application Data
(60) Provisional application No. 63/261,048, filed on Sep. 9, 2021.

(51) Int. Cl. C12Q 1/6886

(52) U.S. Cl. CPC C12Q 1/6886; C12Q 2600/106; C12Q 2600/156

(57) ABSTRACT

The present disclosure relates to methods of identifying, selecting, monitoring, and/or treating a cancer patient with a STING agonist.

This application claims the benefit of and priority to U.S. Provisional Application No. 63/261,048 filed Sep. 9, 2021 entitled “BIOMARKERS FOR STING AGONISTS AND METHODS OF USING THE SAME,” the contents of which are expressly incorporated herein by reference in their entirety.

The innate immune system provides the initial line of defense against infectious pathogens. Studies have shown that the stimulator-of-interferon-genes (STING) protein plays a central role in this response by mediating type I interferon (IFNα and IFNβ) production through both NFk-B and IRF3 transcription pathways in response to intracellular double stranded DNA, intracellular pathogens, and mitochondrial damage (Ishikawa et al. (2008) Nature 455:674-678; Burdette et al. (2013) Nat. Immunol. 14:19-26; Zhong et al. (2008) Immunity 29:538-550; Sun et al. (2009) Proc. Natl. Acad. Sci. USA 106:8653-8658). The innate immune system also plays a role in both pro- and antitumor immunity (Rakoff-Nahoum et al. (2009) Nat. Rev. Cancer. 9:57-63; Rakoff-Nahoum et al. (2008) Biochemistry (Mosc). 73:555-561). It has been demonstrated that intratumoral administration of STING agonists in the tumor microenvironment can initiate an antitumor immune T cell response through the cyclic guanosine monophosphate-adenosine monophosphate synthase-stimulator of interferon genes (cGAS-STING) pathway (Woo et al. (2014) Immunity. 41:830-842; Deng et al. (2014) Immunity. 41:843-852). As such, STING and its activation has emerged as a promising therapeutic target for oncology.

Cancer cell nuclei and mitochondria are prone to release genomic contents into the cytosol in the form of double stranded (ds) DNA. This dsDNA is recognized by cyclic GMP-AMP synthase (cGAS), which upon binding to dsDNA, undergoes a conformational change which facilitates the generation of cyclic Gp(2′,5′)Ap(3′,5′) (cGAMP). STING (also referred to as transmembrane protein 173 or TMEM173) is a transmembrane protein that undergoes a conformational change when binding to cyclic dinucleotides (CDNs) such as cGAMP, thus acting as direct sensor of CDNs in both cancer cells and antigen-presenting cells (Shih et al. (2018) Biophys. J. 114 (1): 32-39). Activation of STING by CDNs is associated with the downstream production of many proinflammatory cytokines and chemokines, such as INFβ, as well as the induction of antiviral genes such as type I IFNs and IFN-stimulated genes (ISG).

STING typically consists of an N-terminal transmembrane region, a C-terminal region that includes the dimerization domain, and a carboxyterminal tail. STING variants have evolved to distinguish noncanonical CDNs produced by mammalian cyclic-GMP-AMP synthase from conventional (3′-5′) CDNs produced primarily by bacteria (Burdette et al. (2011) Nature 478:515-518; Corrales et al. (2015) Cell Rep. 11:1018-1030; Diner et al. (2013) Cell Reports 3:1355-1361).

STING agonists as potential therapeutic agents have been reported and consist mostly of either CDNs analogues or non-nucleotidyl small molecule agonists. Synthetic or naturally derived CDNs analogues function by locking STING in an active conformation (Motedayen et al. (2020) J. Clin. Med. 9, 3323). Multiple STING agonists are in clinical trials, including ADU-S100, BI 1387446, MK-1454, MK-2118, BMS-986301, CDK-002, GSK-3745417, idronoxil, SB-11285, IMSA-101, SNX-281, SYNB-1891, TAK-676, DMXAA, FAA, CMA, α-Mangostin, BNBC, DSDP, diABZI, a bicyclic benzamide, a benzothiophene, MSA 2, SR-717, MAVU-104, TTI-10001, SRCB-0001, CRD 5500, ALG-031048, JNJ-′6196, IACS-8803, IACS-8779, NZ IO STING, OX-401, SITX-799, STACT-TREX1, XMT-2056, ONM-500, MV-626, PF 06928215, DN-15089, HH18202, STI-001, and E7766 or a pharmaceutically acceptable salt thereof. E7766 is a CDN analogue from the novel class of macrocycle-bridged STING agonists (MBSAs). STING agonists have been evaluated or considered for the treatment of melanoma, head and neck squamous cell carcinoma (HNSCC), upper aerodigestive tract cancers, breast cancer, colorectal cancer, and/or tumors including lymphomas. STING agonists have also been evaluated when combined with an additional therapy, e.g. an anti-PD-1 antibody, such as the treatment regimen described in PCT/US2019/034933 (published as WO 2019/232392A1) which is incorporated by reference herein.

Despite the established anti-tumor effects of STING agonists in various preclinical models, many early clinical studies have failed to demonstrate similar anti-tumor efficacy (Meric-Bernstam et al., J. Clin. Oncol. 2019, 37, 2507-2507; Harrington et al., Ann. Oncol. 2018, 29, viii712). One of the challenges facing development of STING agonists is the effect of human genetic variation in proteins involved in the cGAS-STING pathway and their ability to mediate anti-tumor responses. Single nucleotide variants (SNVs) in innate immune receptors have been found to decrease the production of type 1 interferons and cytokines (Yi et al. PloS one 8.10 (2013): e77846). Thus, genetic analysis of patients prior to treatment may be advantageous to identify patients who could respond better to STING agonists and therefore benefit from treatment.

The present disclosure relates to methods of identifying, selecting, monitoring, and/or treating a cancer patient with a STING agonist based on the patient's genotype. In some embodiments, the present disclosure relates to methods of determining a patient's genotype to identify cancer patients suitable for treatment with a STING agonist. In some embodiments, a patient that is identified as suitable for treatment with a STING agonist is administered a therapeutically effective amount of a STING agonist, such as E7766 or a pharmaceutically acceptable salt thereof. In some embodiments, a patient that is identified as suitable for treatment with a STING agonist is also administered an additional therapy, such as a checkpoint inhibitor.

In some embodiments, the present disclosure provides methods of treating a cancer in a patient, comprising administering a therapeutically effective amount of a STING agonist to the patient who carries: (i) reference or heterozygous DNA sequences for TMEM173 V48V (rs7447927); and (ii) reference or heterozygous for TLR6 S249P (rs5743810); or reference for TLR10 I775V (rs4129009). In some embodiments, the present disclosure provides use of a patient's genotype for single nucleotide variant(s) rs7447927, rs5743810, and/or rs4129009 as a biomarker in the manufacture of a medicament for treating a cancer in the patient. In some embodiments, the present disclosure provides use of a patient's genotype for single nucleotide variant(s) rs7447927, rs5743810, and/or rs4129009 as a biomarker for treating a cancer in the patient. In some embodiments, the treating comprises administering a therapeutically effective amount of a STING agonist to the patient who carries: (i) reference or heterozygous DNA sequences for TMEM173 V48V (rs7447927); and (ii) reference or heterozygous for TLR6 S249P (rs5743810); or reference for TLR10 I1775V (rs4129009).

In some embodiments, the present disclosure provides methods of treating a cancer in a patient, comprising: (a) determining that the patient carries: (i) reference or heterozygous DNA sequences for TMEM173 V48V (rs7447927); and (ii) reference or heterozygous DNA sequences for TLR6 S249P (rs5743810); or reference DNA sequences for TLR10 I1775V (rs4129009); and (b) administering a therapeutically effective amount of a STING agonist to the patient. In some embodiments, the present disclosure provides use of a patient's genotype for single nucleotide variant(s) rs7447927, rs5743810, and/or rs4129009 as a biomarker in the manufacture of a medicament for treating a cancer in the patient. In some embodiments, the present disclosure provides use of a patient's genotype for single nucleotide variant(s) rs7447927, rs5743810, and/or rs4129009 as a biomarker for treating a cancer in the patient. In some embodiments, the treating comprises: (a) determining that the patient carries: (i) reference or heterozygous DNA sequences for TMEM173 V48V (rs7447927); and (ii) reference or heterozygous DNA sequences for TLR6 S249P (rs5743810); or reference DNA sequences for TLR10 I775V (rs4129009); and (b) administering a therapeutically effective amount of a STING agonist to the patient.

In some embodiments, the present disclosure provides methods of identifying a cancer patient suitable for treatment with a STING agonist, comprising: (a) determining that the patient carries: (i) reference or heterozygous DNA sequences for TMEM173 V48V (rs7447927); and (ii) reference or heterozygous DNA sequences for TLR6 S249P (rs5743810); or reference DNA sequences for TLR10 I775V (rs4129009); and (b) identifying the patient as suitable for treatment with a STING agonist. In some embodiments, the present disclosure provides use of a patient's genotype for single nucleotide variant(s) rs7447927, rs5743810, and/or rs4129009 as a biomarker in the manufacture of a composition for identifying a cancer patient suitable for treatment with a STING agonist. In some embodiments, the present disclosure provides use of a patient's genotype for single nucleotide variant(s) rs7447927, rs5743810, and/or rs4129009 as a biomarker for identifying a cancer patient suitable for treatment with a STING agonist. In some embodiments, the identifying comprises: (a) determining that the patient carries: (i) reference or heterozygous DNA sequences for TMEM173 V48V (rs7447927); and (ii) reference or heterozygous DNA sequences for TLR6 S249P (rs5743810); or reference DNA sequences for TLR10 I775V (rs4129009); and (b) identifying the patient as suitable for treatment with a STING agonist.

The present disclosure provides, in other embodiments, a STING agonist for use in treating a cancer in a patient. In some embodiments, the treating comprises administering a therapeutically effective amount of the STING agonist to the patient who carries: (i) reference or heterozygous DNA sequences for TMEM173 V48V (rs7447927); and (ii) reference or heterozygous DNA sequences for TLR6 S249P (rs5743810); or reference DNA sequences for TLR10 I1775V (rs4129009).

In some embodiments, the methods and uses described herein comprise obtaining a biological sample from the patient, and determining from the sample the patient's genotype for single nucleotide variant(s) rs7447927, rs5743810, and/or rs4129009. In some embodiments, the biological sample comprises a buccal sample, a blood sample, and/or a tumor sample. In some embodiments, the biological sample is obtained from the patient by a buccal swab, a phlebotomy, and/or a tumor biopsy. In some embodiments, treatment decisions, e.g., whether to administer a STING agonist such as E7766 or a pharmaceutically acceptable salt thereof, are based on the determination of whether single nucleotide variant(s) rs7447927, rs5743810, and/or rs4129009 are present in the sample.

In some embodiments, the patient's genotype for single nucleotide variant(s) rs7447927, rs5743810, and/or rs4129009 is determined by DNA variant testing. In some embodiments, the DNA variant testing comprises next generation sequencing (NGS), polymerase chain reaction (PCR), and/or pyrosequencing. In some embodiments, the patient is reference for rs7447927 and reference for rs5743810. In some embodiments, the patient is reference for rs7447927 and heterozygous for rs5743810. In some embodiments, the patient is heterozygous for rs7447927 and reference for rs5743810. In some embodiments, the patient is heterozygous for rs7447927 and heterozygous for rs5743810. In some embodiments, the patient is reference for rs7447927 and reference for rs4129009. In some embodiments, the patient is heterozygous for rs7447927 and reference for rs4129009.

In some embodiments, the patient has or is suspected of having a cancer of the upper aerodigestive tract. In some embodiments, the cancer comprises a cancer of the lip, buccal mucosa, floor of mouth, oral cavity, hard palate, base of tongue, oral tongue, tonsil, oropharynx, salivary gland, gallbladder, esophagus, stomach, and/or biliary tract. In some embodiments, the cancer comprises a head and neck squamous cell carcinoma (HNSCC). In some embodiments, the cancer comprises an esophageal cancer. In some embodiments, the cancer is a recurrent cancer. In some embodiments, the cancer is a metastatic cancer.

In some embodiments, the STING agonist comprises one or more of E7766 or a pharmaceutically acceptable salt thereof, ADU-S100, BI 1387446, MK-1454, MK-2118, BMS-986301, CDK-002, GSK-3745417, idronoxil, SB-11285, IMSA-101, SNX-281, SYNB-1891, TAK-676, DMXAA, FAA, CMA, α-Mangostin, BNBC, DSDP, diABZI, a bicyclic benzamide, a benzothiophene, MSA-2, SR-717, MAVU-104, TTI-10001, SRCB-0001, CRD 5500, ALG-031048, JNJ-′6196, IACS-8803, IACS-8779, NZ-IO-STING, OX-401, SITX-799, STACT-TREX1, XMT-2056, ONM-500, MV-626, PF-06928215, DN-15089, HH18202, and/or STI-001. In some embodiments, the STING agonist comprises one or more of E7766 or a pharmaceutically acceptable salt thereof, ADU-S100, BI 1387446, MK-1454, MK-2118, BMS-986301, CDK-002, GSK-3745417, idronoxil, SB-11285, IMSA-101, SNX-281, SYNB-1891, and/or TAK-676. In some embodiments, the STING agonist comprises a diammonium salt of E7766.

In some embodiments, the STING agonist is administered to the patient intratumorally. In some embodiments, the STING agonist is administered to the patient via an intra-tumoral injection. In some embodiments, the injection is administered into a solid tumor, e.g., wherein the tumor has a long-axis diameter of at least 1 centimeter. In some embodiments, the injection is administered into a lymph node, e.g., wherein the lymph node has a short-axis diameter of at least 1.5 centimeters. In some embodiments, the STING agonist is administered systemically. In some embodiments, the STING agonist is administered to the patient via an intravenous infusion. In some embodiments, the STING agonist is administered to the patient via subcutaneously.

In some embodiments, the STING agonist is administered to the patient in combination with at least one additional therapy. In some embodiments, the at least one additional therapy comprises administering one or more checkpoint inhibitors. In some embodiments, the checkpoint inhibitor targets PD1, PDL1, and/or CTLA4. In some embodiments, the checkpoint inhibitor comprises a programmed death-1 pathway (PD1) inhibitor. In some embodiments, the PD1 inhibitor comprises an anti-PD1 antibody. In some embodiments, the anti-PD1 antibody comprises pembrolizumab, nivolumab, and/or spartalizumab. In some embodiments, the PD1 inhibitor comprises an anti-PDL1 antibody. In some embodiments, the checkpoint inhibitor comprises a cytotoxic T-lymphocyte-associated antigen 4 pathway (CTLA4) inhibitor. In some embodiments, the CTLA4 inhibitor comprises an anti-CTLA4 antibody. In some embodiments, the anti-CTLA4 antibody comprises ipilimumab.

In some embodiments, the STING agonist is administered to the patient in combination with at least one additional therapy. In some embodiments, the additional therapy is administered to the subject before administration of the STING agonist. In some embodiments, the additional therapy is administered to the subject simultaneously with the administration of the STING agonist. In some embodiments, the additional therapy is administered to the subject following administration of the STING agonist. In some embodiments, the additional therapy is administered to the patient intratumorally. In some embodiments, the additional therapy is administered to the patient via an intra-tumoral injection. In some embodiments, the injection is administered into a solid tumor, e.g., wherein the tumor has a long-axis diameter of at least 1 centimeter. In some embodiments, the injection is administered into a lymph node, e.g., wherein the lymph node has a short-axis diameter of at least 1.5 centimeters. In some embodiments, the additional therapy is administered systemically. In some embodiments, the additional therapy is administered to the patient via an intravenous infusion.

In some embodiments, treatment with a STING agonist (e.g., any of the exemplary STING agonists described or incorporated by reference herein) reduces or inhibits growth of a tumor in the patient, as compared to the growth of the tumor prior to treatment. In some embodiments, treatment reduces the size of a tumor in the patient, as compared to the size of the tumor prior to treatment. In some embodiments, treatment reduces the size of a tumor in the patient by about 5%, 10%, 15%, 20%, or more, as compared to the size of the tumor prior to treatment. In some embodiments, treatment reduces the size of a tumor in the patient by about 20% or more, as compared to the size of the tumor prior to treatment. In some embodiments, treatment increases or decreases the expression or activity of at least one protein in the patient, as compared to the expression or activity of the same protein(s) prior to treatment. In some embodiments, the at least one protein comprises a cytokine and/or a chemokine. In some embodiments, the at least one protein comprises IFNα, IFNβ, IFNγ, IP-10, MCP-1, MIP-1b, IL-6, and/or TNFα.

### BRIEF DESCRIPTION OF THE DRAWINGS

FIG. 1 shows a hazard ratio plot of life expectancy for patients with upper aerodigestive tract (oral cavity, oropharynx, esophageal, gastric, biliary tract) tumors who carried reference/heterozygous TLR6 S249 and/or reference TLR10 I775, in combination with Neandertal-associated STING1 rs7447927.

FIG. 2A-B shows circulating cytokine levels in a patient after first administration of the STING agonist Compound 1.

FIG. 3 shows changes in gene expression in white blood cells from a patient administered the first dose of the STING agonist Compound 1.

### DETAILED DESCRIPTION

The following detailed description and examples illustrate certain embodiments of the present disclosure. Those of skill in the art will recognize that there are numerous variations and modifications of this disclosure that are encompassed by its scope. Accordingly, the description of certain embodiments should not be deemed to limit the scope of the present disclosure.

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

All references cited herein, including, but not limited to, published and unpublished patent applications, granted patents, and literature references, are incorporated herein by reference and are hereby made a part of this specification. To the extent a cited reference conflicts with the disclosure herein, the specification shall control.

As used herein, the singular forms of a word also include the plural form, unless the context clearly dictates otherwise; as examples, the terms “a,” “an,” and “the” are understood to be singular or plural. By way of example, “an element” means one or more element. The term “or” shall mean “and/or” unless the specific context indicates otherwise.

The term “agent,” as used herein, refers to a chemical compound, a mixture of chemical compounds, a biological macromolecule (e.g., an antibody), an extract made from biological materials, or a combination or conjugate thereof. The term “therapeutic agent” refers to an agent that is capable of providing a therapeutic benefit, e.g., modulating a biological process and/or providing biological activity. In some embodiments, an agent is a STING agonist.

The term “STING agonist” refers to an agent that may act directly on STING or an upstream or downstream component of the cGAS-STING pathway to provide an agonist effect. Such agonist effects may include increasing the concentration of CDNs, such as cGAMP. In some embodiments, a STING agonist is a synthetic or naturally occurring CDN analog which increases STING activation by locking STING in an active conformation. In some embodiments, a STING agonist increases STING activation indirectly, for example, by preventing cGAMP degradation by ENPP1. In some embodiments, a STING agonist increases the concentration of cytokines downstream of STING in the cGAS-STING pathway (e.g., INFβ). In some embodiments, a STING agonist increases the expression of antiviral genes (e.g., INFs).

Treatment of a patient with a STING agonist may result in a biological response. In some embodiments, the biological response is a reduction in tumor growth rate or tumor volume. In some embodiments, the biological response is a reduction in a symptom of cancer. In some embodiments, a STING agonist is capable of binding to human STING or a functional variant or fragment thereof and providing an agonist effect such as increasing or potentiating STING activity and/or stabilizing circulating STING (e.g., human STING). In some embodiments, a STING agonist is capable of binding to an upstream or downstream molecule in the cGAS-STING pathway to provide an agonist effect. In some embodiments, the STING agonist is a small molecule. In some embodiments, the STING agonist is a biologic molecule, such as an antibody or antigen-binding fragment. In some embodiments, the STING agonist is a conjugate of an antibody and a small molecule.

As used herein, “functional variant” or “fragment” herein refers to a peptide or nucleic acid that differs from a parent polypeptide or nucleic acid by one or more amino acid or nucleic acid deletions, substitutions or additions, yet retains one or more specific functions or biological activities of the parent molecule. Nucleic acid substitutions include alterations in which a single nucleic acid is replaced with a different nucleic acid. Such substitutions may be classified as “synonymous variant”, in which case the amino acid residue encoded by the nucleic acid sequence is unchanged by the substitution. Substitutions may be classified as “missense variant”, in which case the amino acid residue encoded by the nucleic acid sequence is changed by the substitution. Also encompassed within the term variant when used with reference to a polynucleotide or polypeptide, refers to a polynucleotide or polypeptide that can vary in primary, secondary, or tertiary structure, as compared to a reference polynucleotide or polypeptide, respectively (e.g., as compared to a wild-type polynucleotide or polypeptide).

“E7766” as used herein, refers to a macrocycle-bridged STING agonist (MBSA) as described in Kim et al., ChemMedChem. 2021 Jun. 7; 16 (11): 1740-1743 and PCT/US2020/040515 (published as WO2021003279A1), which are incorporated by reference herein. The structure is shown below:

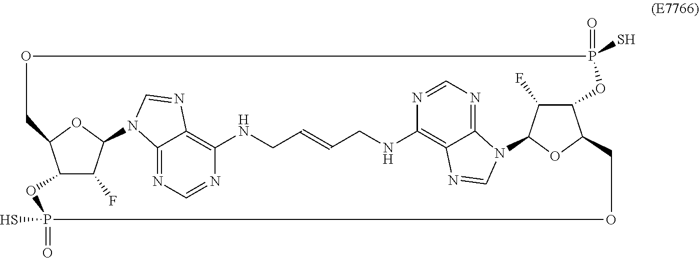 [SMILES: O=[P@]1(S)OC[C@H]2O[C@@H]3[C@H](F)[C@@H]2O[P@](=O)(S)OC[C@H]2O[C@H]([C@H](F)[C@@H]2O1)n1cnc2c(ncnc21)NC/C=C/CNc1ncnc2c1ncn23]

E7766 is also referred to as (1R,3R,15E,28R,29R,30R,31R,34R,36R,39S,41R)-29,41-Difluoro-34,39-bis(sulfanyl)-2,33,35,38,40,42-hexaoxa-4,6,9,11,13,18,20,22,25,27-decaaza-34k⁵,39k⁵-diphosphaoctacyclo [28.6.4.1^{3,36}.1^{28,31}.0^{4,8}.0^{7,12}.0^{19,24}.0^{23,27}]dotetraconta-5,7,9,11,15,19,21,23,25-nonaene-34,39-dione. If there exist any discrepancies as between this chemical name and the structure given above, the structure given above will control. Compound 1, which is the diammonium salt of E7766, has a molecular weight of 780.7. In the examples reported herein, Compound 1, the diammonium salt, was used.

The term “pharmaceutically acceptable,” as used herein, means approved or approvable by a regulatory agency of the Federal or a state government or listed in the U.S. Pharmacopeia or other generally recognized pharmacopeia, for use in animals, and more particularly in humans.

A “pharmaceutically acceptable salt,” as used herein, is a salt that retains a desired biological activity of the parent compound to which it is added and does not impart undesired toxicological effects. Examples of such salts are: (a) acid addition salts formed with inorganic acids, for example, hydrochloric acid, hydrobromic acid, sulfuric acid, phosphoric acid, nitric acid and the like; and salts formed with organic acids, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine. See, e.g., Haynes et al. “Commentary: Occurrence of Pharmaceutically Acceptable Anions and Cations in the Cambridge Structural Database,” J Pharmaceutical Sciences, Vol. 94, No. 10 (2005), and Berge et al. “Pharmaceutical Salts,” J Pharmaceutical Sciences, Vol. 66, No. 1 (1977), which are incorporated by reference herein.

A “pharmaceutical excipient” refers to a material such as an adjuvant, a carrier, pH-adjusting and buffering agents, tonicity adjusting agents, wetting agents, preservative, and the like.

A “pharmaceutical composition” refers to a preparation which is in such form as to permit administration and subsequently provide the intended biological activity of the active ingredient(s) and/or to achieve a therapeutic effect, and which contains no additional components which are unacceptably toxic to a patient to which the formulation would be administered. The pharmaceutical composition may be sterile.

An “effective amount” of, e.g., a STING agonist, e.g., E7766 or a pharmaceutically acceptable salt thereof, is an amount sufficient to perform a specifically stated purpose, for example to produce a therapeutic effect after administration to a patient, such as a reduction in tumor growth rate or tumor volume, a reduction in a symptom of cancer, or some other indicia of treatment efficacy. The term “therapeutically effective amount” refers to an amount of, e.g., a STING agonist, e.g., E7766 or a pharmaceutically acceptable salt thereof, effective to provide a desired therapeutic effect, such as to treat a disease or disorder in a patient. In the case of cancer, a therapeutically effective amount of a STING agonist, e.g., E7766 or a pharmaceutically acceptable salt thereof, may measurably alter (e.g., increase or decrease) one or more clinically measured parameters of the cancer (e.g., one or more phenotypes and/or biomarkers regulated by STING), reduce the number of cancer cells, reduce tumor size, inhibit (e.g., slow or stop) tumor metastasis, inhibit (e.g., slow or stop) tumor growth, and/or relieve one or more symptoms. A “prophylactically effective amount” refers to an amount effective, at dosages and for periods of time necessary, to achieve the desired prophylactic result. Typically, since a prophylactic dose is used in patients prior to or at an earlier stage of disease, the prophylactically effective amount will be less than the therapeutically effective amount.

As used herein, the term “treat” or “treatment” or “therapeutic” (and grammatically related terms) refers to any improvement of any consequence of disease, such as prolonged survival, delayed progression of disease (e.g. delayed or reduced growth in a tumor volume), improvement (e.g., a reduction in tumor volume), less morbidity, and/or a lessening of side effects which result from an alternative therapeutic modality. Full eradication of a disease or a symptom or consequence thereof is encompassed but not required for a treatment act. Treatment may refer to the administration of one or more agents, e.g., a STING agonist (e.g., E7766 or a pharmaceutically acceptable salt thereof) to a patient, e.g., a cancer patient. The treatment can be to prevent, cure, heal, alleviate, relieve, alter, remedy, ameliorate, palliate, improve or affect the disease, one or more symptoms or consequences of the disease, or the predisposition toward the disease, e.g., cancer.

The terms “sample” and “biological sample” refer to any biological specimen from a subject or patient, e.g., a human patient. Exemplary biological samples include but are not limited to cells, tissues, and body fluids such as blood, serum, plasma, bowel fluid, as well as samples of stool, organs, and veins. A biological sample is generally representative of any other sample taken from the same source, and can be stored such that the stored sample remains biologically equivalent to the sample at the time of its collection. In some embodiments, the biological sample comprises a buccal sample, a blood sample, and/or a tumor sample. In some embodiments, the biological sample is obtained from a patient by a buccal swab, a phlebotomy, and/or a tumor biopsy.

In some embodiments, a biological response is detected or monitored in a patient or in a sample from a patient. The term “biological response,” as used herein, refers to a response in a patient (or sample) after administration of (or contact with) an agent, such as a STING agonist (e.g., E7766 or a pharmaceutically acceptable salt thereof). A biological response can include any response related to, for example, cell signaling or signal transduction (e.g., phosphorylation of a protein kinase), gene transcription, protein expression, toxicity, cytokine release, cell proliferation, cell motility or morphology, cell growth arrest, and/or cell death (e.g., apoptosis).

As used herein, the term “cytokine” refers to any molecule produced by a cell that influences the function of other cells to mediate an immune response. The term “chemokine,” as used herein, refers to a type of cytokine that induces chemotaxis and/or specifically influences the migration of cells to sites of infection/inflammation. Exemplary cytokines and/or chemokines include but are not limited to IFNα, IFNβ, IFNγ, IP-10, MCP-1, MIP-1b, IL-6, and/or TNFα.

As used herein, the term “gene” refers to a nucleic acid molecule comprising an open reading frame encoding a polypeptide. Natural allelic variations can typically result in 1-5% variance in the nucleotide sequence of a given gene. Alternative alleles can be identified by sequencing the gene of interest in a number of different individuals. This can be readily carried out by using methods known in the art.

As used herein, “allele” refers to a nucleic acid sequence on one of the two chromosomes for a gene locus.

As used herein, the term “wild-type allele”, “reference allele” or “standard allele” refers to the nucleic acid sequence of the allele most frequently found in the human population. In some embodiments, a patient is considered to be “reference” for a gene locus if both alleles are wild-type.

As used herein, “wild-type” and “WT” may be used interchangeably herein to refer to a wild-type allele, gene, gene locus, or gene product.

As used herein, the term “allelic variant” or “variant” refers to a non-wild-type allele, gene, gene locus, or gene product.

As used herein, “DNA variant testing” refers to the use of DNA sequencing to determine the sequence of an allele and compare to a wild-type allele or to directly detect the presence of altered nucleotides in a given reference sequence relative to a wild-type sequence. In some embodiments, DNA variant testing comprises the use of next generation sequencing (NGS). In some embodiments, DNA variant testing comprises the use of polymerase chain reaction (PCR). In some embodiments, DNA variant testing comprises the use of pyrosequencing. Other DNA sequencing methods are known in the art and may be used.

As used herein, “genotype” refers to the two alleles present for a particular gene locus in a subject.

As used herein, a “single nucleotide variant” (SNV) is an allelic variant that differs from a reference allele by a single nucleotide at a specific genomic position. In some embodiments, a single nucleotide variant can be identified by a reference number (Reference SNP (rs)).

As used herein, a patient is said to be “reference” for an allele in a gene locus if the patient has two identical wild-type type alleles for that gene locus.

As used herein, a patient is said to be “heterozygous” for an allele in a gene locus if the patient has two different alleles for a gene locus and one of the alleles is a wild-type allele.

As used herein, a patient is said to be “homozygous” for an allele in a gene locus if the patient has two identical, non-wild-type alleles for that gene locus.

As used herein, the term “human STING gene” (STING gene also referred to as “TMEM173”) refers to a nucleic acid molecule comprising an open reading frame encoding human STING or any functional variants or fragments thereof (i.e., variants and fragments are encompassed unless the context indicates that the term is used to refer to the wild-type gene only). As used herein, “wild-type human STING gene” comprises or consists of the nucleotide sequence of SEQ ID NO: 2.

Allelic variants of the human STING gene comprise or consists of a nucleotide sequence that is not SEQ ID NO: 2. In some embodiments, allelic variants of the human STING gene comprise or consist of the nucleotide sequence of SEQ ID NO: 3 or SEQ ID NO: 4. In some embodiments, allelic variants of the human STING may comprise or consist of the reference sequence TMEM173 (rs7447927).

As used herein, the term “human STING” refers to any native form of the human stimulator-of-interferon-genes (STING) protein. The term encompasses full-length human STING, as well as any form of human STING that may result from cellular processing. The term also encompasses functional variants or fragments of human STING, including but not limited to allelic variants, splice variants, and isoforms that retain one or more biologic functions of human STING (i.e., variants and fragments are encompassed unless the context indicates that the term is used to refer to the wild-type protein only).

As used herein, the term “wild-type human STING” refers to the gene product of the wild-type allele found at the human STING locus. As used herein, the wild-type human STING is encoded by the nucleotide sequence of SEQ ID NO: 2. As used herein, the wild-type human STING is the amino acid sequence of SEQ ID NO: 1.

As used herein, the term “allelic variant of human STING” refers to the gene product of a non-wild-type allele found at the human STING locus and encompasses all allelic variants of human STING. Exemplary allelic variants of human STING are described in Patel and Lin ((2019) Genes & Immunity 20:82-89), which is incorporated herein by reference for the disclosure and sequences of such variants. Exemplary allelic variants of human STING include but are not limited to HAQ (R71H-G230A-R293Q), AQ (G230A-R293Q), Q293, and H232 (Patel and Lin (2019) Genes & Immunity 20:82-89). In some embodiments, an allelic variant of human STING is a synonymous variant. In some embodiments, a synonymous variant of human STING may be encoded by TMEM173 (rs7447927).

Exemplary STING sequences are set forth in Table 1.

As used herein, the term “human TLR6 gene” (TLR6 gene also referred to as “TLR6”) refers to a nucleic acid molecule comprising an open reading frame encoding human Toll-like receptor 6 protein or any functional variants or fragments thereof (i.e., variants and fragments are encompassed unless the context indicates that the term is used to refer to the wild-type gene only). As used herein, the wild-type human TLR6 gene comprises or consists of the nucleotide sequence of SEQ ID NO: 6.

Allelic variants of the human TLR6 gene comprise or consists of a nucleotide sequence that is not SEQ ID NO: 6. In some embodiments, allelic variants of the human TLR6 gene comprise or consist of the nucleotide sequence of SEQ ID NO: 8. In some embodiments, allelic variants of the human TLR6 gene may comprise or consist of the reference sequence TLR6 (rs5743810).

As used herein, the term “human TLR6” refers to any native form of the human TLR6 protein. The term encompasses full-length human TLR6, as well as any form of human TLR6 that may result from cellular processing. The term also encompasses functional variants or fragments of human TLR6, including but not limited to allelic variants, splice variants, and isoforms that retain one or more biologic functions of human TLR6 (i.e., variants and fragments are encompassed unless the context indicates that the term is used to refer to the wild-type protein only).

As used herein, the term “wild-type human TLR6” refers to the gene product of the wild-type allele found at the human TLR6 locus. As used herein, the wild-type human TLR6 is encoded by the nucleotide sequence of SEQ ID NO: 6. As used herein, the wild-type human TLR6 is the amino acid sequence of SEQ ID NO: 5.

As used herein, the term “allelic variant of human TLR6” refers to the gene or gene product of a non-wild-type allele found at the human TLR6 locus and encompasses all allelic variants of human TLR6. In some embodiments, the human TLR6 is an allelic variant of human TLR6 comprising a S249P mutation. In some embodiments, the allelic variant of human TLR6 comprising a S249P mutation comprises SEQ ID NO: 7. In some embodiments, an allelic variant of human TLR6 may be encoded by TLR6 (rs5743810).

Exemplary TLR6 sequences are set forth in Table 2.

As used herein, the term “human TLR10 gene” (TLR10 gene also referred to as “TLR10”) refers to a nucleic acid molecule comprising an open reading frame encoding human Toll-like receptor 10 protein or any functional variants or fragments thereof (i.e., variants and fragments are encompassed unless the context indicates that the term is used to refer to the wild-type gene only). In some embodiments, the wild-type human TLR10 gene comprises or consists of the nucleotide sequence of SEQ ID NO: 10.

Allelic variants of the human TLR10 gene comprise or consists of a nucleotide sequence that is not SEQ ID NO: 10. In some embodiments, allelic variants of the human TLR10 gene comprise or consist of the nucleotide sequence of SEQ ID NO: 12. In some embodiments, allelic variants of the human TLR10 gene may comprise or consist of the reference sequence TLR10 (rs4129009).

As used herein, the term “human TLR10” refers to any native form of the human TLR10 protein. The term encompasses full-length human TLR10, as well as any form of human TLR10 that may result from cellular processing. The term also encompasses functional variants or fragments of human TLR10, including but not limited to allelic variants, splice variants, and isoforms that retain one or more biologic functions of human TLR10 (i.e., variants and fragments are encompassed unless the context indicates that the term is used to refer to the wild-type protein only).

As used herein, the term “wild-type human TLR10” refers to the gene product of the wild-type allele found at the human TLR10 locus. As used herein, the wild-type human TLR10 is encoded by the nucleotide sequence of SEQ ID NO: 10. As used herein, the wild-type human TLR10 is the amino acid sequence of SEQ ID NO: 9.

As used herein, the term “allelic variant of human TLR10” refers to the gene or gene product of a non-wild-type allele found at the human TLR10 locus and encompasses all allelic variants of human TLR10. In some embodiments, the human TLR10 is an allelic variant of human TLR10 comprising a 1775V mutation. In some embodiments, the allelic variant of human TLR10 comprising a 1775V mutation comprises SEQ ID NO: 11. In some embodiments, an allelic variant of human TLR10 may be encoded by TLR10 (rs4129009).

Exemplary TLR10 sequences are set forth in Table 3.

Residues in two or more polypeptides are said to “correspond” if the residues occupy an analogous position in the polypeptide structures. Analogous positions in two or more polypeptides can be determined by aligning the polypeptide sequences based on amino acid sequence or structural similarities. Those skilled in the art understand that it may be necessary to introduce gaps in either sequence to produce a satisfactory alignment. Unless otherwise indicated, residue positions are specified with reference to the amino acid numbering in a wild-type human STING (SEQ ID NO: 1). For example, a variant of human STING (e.g., an allelic variant of human STING) may comprise a modification to the arginine at position 232, as numbered from the N-terminus of SEQ ID NO: 1. In this example, the arginine at position 232 of SEQ ID NO: 1 is denoted “R232.” If the arginine at position 232 of SEQ ID NO: 1 is replaced, for example, with a histidine, the histidine modification at position 232 is denoted “R232H.”

TABLE 1
STING Alleles.

| Name | SEQ ID NO | Sequence Type | Sequence |
|---|---|---|---|
| Human TMEM173 (WT) | 1 | Amino acid | MPHSSLHPSIPCPRGHGAQKAALVLLSACLVTLWGLGEPPEHTLRYLVLHL |
| | | | ASLQLGLLLNGVCSLAEELRHIHSRYRGSYWRTVRACLGCPLRRGALLLLSI |
| | | | YFYYSLPNAVGPPFTWMLALLGLSQALNILLGLKGLAPAEISAVCEKGNFNV |
| | | | AHGLAWSYYIGYLRLILPELQARIRTYNQHYNNLLRGAVSQRLYILLPLDCG |
| | | | VPDNLSMADPNIRFLDKLPQQTGDRAGIKDRVYSNSIYELLENGQRAGTCV |
| | | | LEYATPLQTLFAMSQYSQAGFSREDRLEQAKLFCRTLEDILADAPESQNNC |
| | | | RLIAYQEPADDSSFSLSQEVLRHLRQEEKEEVTVGSLKTSAVPSTSTMSQE |
| | | | PELLISGMEKPLPLRTDFS |
| Human TMEM173 (WT) | 2 | Nucleic acid | atgccccactccagcctgcatccatccatcccgtgtcccaggggtcacggggcccagaaggcagccttg |
| | | | gttctgctgagtgcctgcctggtgaccctttgggggctaggagagccaccagagcacactctccggtacct |
| | | | ggtcctccacctagcctccctgcagctgggactgctgttaaacggggtctgcagcctggctgaggagctgc |
| | | | gccacatccactccaggtaccggggcagctactggaggactgtgcgggcctgcctgggctgccccctcc |
| | | | gccgtggggccctgttgctgctgtccatctatttctactactccctcccaaatgcggtcggcccgcccttcactt |
| | | | ggatgcttgccctcctgggcctctcgcaggcactgaacatcctcctgggcctcaagggcctggccccagct |
| | | | gagatctctgcagtgtgtgaaaaagggaatttcaacgtggcccatgggctggcatggtcatattacatcgg |
| | | | atatctgcggctgatcctgccagagctccaggcccggattcgaacttacaatcagcattacaacaacctgc |
| | | | tacggggtgcagtgagccagcggctgtatattctcctcccattggactgtggggtgcctgataacctgagtat |
| | | | ggctgaccccaacattcgcttcctggataaactgccccagcagaccggtgaccgggctggcatcaagga |
| | | | tcgggtttacagcaacagcatctatgagcttctggagaacgggcagcggggggcacctgtgtcctggag |
| | | | tacgccacccccttgcagactttgtttgccatgtcacaatacagtcaagctggctttagccgggaggatagg |
| | | | cttgagcaggccaaactcttctgccggacacttgaggacatcctggcagatgcccctgagtctcagaaca |
| | | | actgccgcctcattgcctaccaggaacctgcagatgacagcagcttctcgctgtcccaggaggttctccgg |
| | | | cacctgcggcaggaggaaaaggaagaggttactgtgggcagcttgaagacctcagcggtgcccagtac |
| | | | ctccacgatgtcccaagagcctgagctcctcatcagtggaatggaaaagcccctccctctccgcacggatt |
| | | | tctcttga |
| Human TMEM173 | 3 | Nucleic acid | atgccccactccagcctgcatccatccatcccgtgtcccaggggtcacggggcccagaaggcagccttg |
| (V48V) C > G | | | gttctgctgagtgcctgcctggtgaccctttgggggctaggagagccaccagagcacactctccggtacct |
| | | | ggtgctccacctagcctccctgcagctgggactgctgttaaacggggtctgcagcctggctgaggagctgc |
| | | | gccacatccactccaggtaccggggcagctactggaggactgtgcgggcctgcctgggctgccccctcc |
| | | | gccgtggggccctgttgctgctgtccatctatttctactactccctcccaaatgcggtcggcccgcccttcactt |
| | | | ggatgcttgccctcctgggcctctcgcaggcactgaacatcctcctgggcctcaagggcctggccccagct |
| | | | gagatctctgcagtgtgtgaaaaagggaatttcaacgtggcccatgggctggcatggtcatattacatcgg |
| | | | atatctgcggctgatcctgccagagctccaggcccggattcgaacttacaatcagcattacaacaacctgc |
| | | | tacggggtgcagtgagccagcggctgtatattctcctcccattggactgtggggtgcctgataacctgagtat |
| | | | ggctgaccccaacattcgcttcctggataaactgccccagcagaccggtgaccgggctggcatcaagga |
| | | | tcgggtttacagcaacagcatctatgagcttctggagaacgggcagcggggggcacctgtgtcctggag |
| | | | tacgccacccccttgcagactttgtttgccatgtcacaatacagtcaagctggctttagccgggaggatagg |
| | | | cttgagcaggccaaactcttctgccggacacttgaggacatcctggcagatgcccctgagtctcagaaca |
| | | | actgccgcctcattgcctaccaggaacctgcagatgacagcagcttctcgctgtcccaggaggttctccgg |
| | | | cacctgcggcaggaggaaaaggaagaggttactgtgggcagcttgaagacctcagcggtgcccagtac |
| | | | ctccacgatgtcccaagagcctgagctcctcatcagtggaatggaaaagcccctccctctccgcacggatt |
| | | | tctcttga |
| Human TMEM173 | 4 | Nucleic acid | atgccccactccagcctgcatccatccatcccgtgtcccaggggtcacggggcccagaaggcagccttg |
| (V48V) C > T | | | gttctgctgagtgcctgcctggtgaccctttgggggctaggagagccaccagagcacactctccggtacct |
| | | | ggttctccacctagcctccctgcagctgggactgctgttaaacggggtctgcagcctggctgaggagctgc |
| | | | gccacatccactccaggtaccggggcagctactggaggactgtgcgggcctgcctgggctgccccctcc |
| | | | gccgtggggccctgttgctgctgtccatctatttctactactccctcccaaatgcggtcggcccgcccttcactt |
| | | | ggatgcttgccctcctgggcctctcgcaggcactgaacatcctcctgggcctcaagggcctggccccagct |
| | | | gagatctctgcagtgtgtgaaaaagggaatttcaacgtggcccatgggctggcatggtcatattacatcgg |
| | | | atatctgcggctgatcctgccagagctccaggcccggattcgaacttacaatcagcattacaacaacctgc |
| | | | tacggggtgcagtgagccagcggctgtatattctcctcccattggactgtggggtgcctgataacctgagtat |
| | | | ggctgaccccaacattcgcttcctggataaactgccccagcagaccggtgaccgggctggcatcaagga |
| | | | tcgggtttacagcaacagcatctatgagcttctggagaacgggcagcggggggcacctgtgtcctggag |
| | | | tacgccacccccttgcagactttgtttgccatgtcacaatacagtcaagctggctttagccgggaggatagg |
| | | | cttgagcaggccaaactcttctgccggacacttgaggacatcctggcagatgcccctgagtctcagaaca |
| | | | actgccgcctcattgcctaccaggaacctgcagatgacagcagcttctcgctgtcccaggaggttctccgg |
| | | | cacctgcggcaggaggaaaaggaagaggttactgtgggcagcttgaagacctcagcggtgcccagtac |
| | | | ctccacgatgtcccaagagcctgagctcctcatcagtggaatggaaaagcccctccctctccgcacggatt |
| | | | tctcttga |

TABLE 2
TLR6 Alleles.

| Name | SEQ ID NO | Sequence Type | Sequence |
|---|---|---|---|
| Human TLR6 (WT) | 5 | Amino acid | MTKDKEPIVKSFHFVCLMIIIVGTRIQFSDGNEFAVDKSKRGLIHVPKDLPLKT |
| | | | KVLDMSQNYIAELQVSDMSFLSELTVLRLSHNRIQLLDLSVFKFNQDLEYLD |
| | | | LSHNQLQKISCHPIVSFRHLDLSFNDFKALPICKEFGNLSQLNFLGLSAMKL |
| | | | QKLDLLPIAHLHLSYILLDLRNYYIKENETESLQILNAKTLHLVFHPTSLFAIQV |
| | | | NISVNTLGCLQLTNIKLNDDNCQVFIKFLSELTRGSTLLNFTLNHIETTWKCL |
| | | | VRVFQFLWPKPVEYLNIYNLTIIESIREEDFTYSKTTLKALTIEHITNQVFLFSQ |
| | | | TALYTVFSEMNIMMLTISDTPFIHMLCPHAPSTFKFLNFTQNVFTDSIFEKCS |
| | | | TLVKLETLILQKNGLKDLFKVGLMTKDMPSLEILDVSWNSLESGRHKENCT |
| | | | WVESIVVLNLSSNMLTDSVFRCLPPRIKVLDLHSNKIKSVPKQVVKLEALQE |
| | | | LNVAFNSLTDLPGCGSFSSLSVLIIDHNSVSHPSADFFQSCQKMRSIKAGDN |
| | | | PFQCTCELREFVKNIDQVSSEVLEGWPDSYKCDYPESYRGSPLKDFHMSE |
| | | | LSCNITLLIVTIGATMLVLAVTVTSLCIYLDLPWYLRMVCQWTQTRRRARNIP |
| | | | LEELQRNLQFHAFISYSEHDSAWVKSELVPYLEKEDIQICLHERNFVPGKSI |
| | | | VENIINCIEKSYKSIFVLSPNFVQSEWCHYELYFAHHNLFHEGSNNLILILLEPI |
| | | | PQNSIPNKYHKLKALMTQRTYLQWPKEKSKRGLFWANIRAAFNMKLTLVTE |
| | | | NNDVKS |
| Human TLR6 (WT) | 6 | Nucleic acid | ccagctcttacctggtagtgtccatcactaagtgtaaagtttggggagaatggagtgtggcagtccgtagaa |
| | | | ggaaaactcagcatgctgatccactcatgtcagtaccattggggtaaaagggacactgcctagctactcat |
| | | | taagtactagttggaactcatcctcactgtttgaatcagagacagagagaggacaaatttctttctgcctttcat |
| | | | cagggagtctattttcctgagacaaagcttgagtgtggcaggtttgccctaacacagttccagttacatttttgt |
| | | | gagtgaagatgctcctggatcctgccattacatttctgtgcttctttgttttcagtggccaagtggatttttcaaatg |
| | | | tatgtgtgtctctgtgggtgttctgatgggcagttgggagaagttatatggatcagtgaagagcttttaaccata |
| | | | cgccattcagtaataaaagttccccaactcagagttttcactcttccaaagaaagaatttggactcatatcaa |
| | | | gatgctctgaagaagaacaaccctttaggatagccactgcaacatcatgaccaaagacaaagaacctat |
| | | | tgttaaaagcttccattttgtttgccttatgatcataatagttggaaccagaatccagttctccgacggaaatga |
| | | | atttgcagtagacaagtcaaaaagaggtcttattcatgttccaaaagacctaccgctgaaaaccaaagtctt |
| | | | agatatgtctcagaactacatcgctgagcttcaggtctctgacatgagctttctatcagagttgacagttttgag |
| | | | actttcccataacagaatccagctacttgatttaagtgttttcaagttcaaccaggatttagaatatttggatttat |
| | | | ctcataatcagttgcaaaagatatcctgccatcctattgtgagtttcaggcatttagatctctcattcaatgatttc |
| | | | aaggccctgcccatctgtaaggaatttggcaacttatcacaactgaatttcttgggattgagtgctatgaagct |
| | | | gcaaaaattagatttgctgccaattgctcacttgcatctaagttatatccttctggatttaagaaattattatataa |
| | | | aagaaaatgagacagaaagtctacaaattctgaatgcaaaaacccttcaccttgtttttcacccaactagttt |
| | | | attcgctatccaagtgaacatatcagttaatactttagggtgcttacaactgactaatattaaattgaatgatga |
| | | | caactgtcaagttttcattaaatttttatcagaactcaccagaggttcaaccttactgaattttaccctcaaccac |
| | | | atagaaacgacttggaaatgcctggtcagagtctttcaatttctttggcccaaacctgtggaatatctcaatatt |
| | | | tacaatttaacaataattgaaagcattcgtgaagaagattttacttattctaaaacgacattgaaagcattgac |
| | | | aatagaacatatcacgaaccaagtttttctgttttcacagacagctttgtacaccgtgttttctgagatgaacatt |
| | | | atgatgttaaccatttcagatacaccttttatacacatgctgtgtcctcatgcaccaagcacattcaagtttttga |
| | | | actttacccagaacgttttcacagatagtatttttgaaaaatgttccacgttagttaaattggagacacttatctt |
| | | | acaaaagaatggattaaaagaccttttcaaagtaggtctcatgacgaaggatatgccttctttggaaatact |
| | | | ggatgttagctggaattctttggaatctggtagacataaagaaaactgcacttgggttgagagtatagtggtg |
| | | | ttaaatttgtcttcaaatatgcttactgactctgttttcagatgtttacctcccaggatcaaggtacttgatcttcac |
| | | | agcaataaaataaagagcgttcctaaacaagtcgtaaaactggaagctttgcaagaactcaatgttgcttt |
| | | | caattctttaactgaccttcctggatgtggcagctttagcagcctttctgtattgatcattgatcacaattcagtttc |
| | | | ccacccatcggctgatttcttccagagctgccagaagatgaggtcaataaaagcaggggacaatccattc |
| | | | caatgtacctgtgagctaagagaatttgtcaaaaatatagaccaagtatcaagtgaagtgttagagggctg |
| | | | gcctgattcttataagtgtgactacccagaaagttatagaggaagcccactaaaggactttcacatgtctga |
| | | | attatcctgcaacataactctgctgatcgtcaccatcggtgccaccatgctggtgttggctgtgactgtgacct |
| | | | ccctctgcatctacttggatctgccctggtatctcaggatggtgtgccagtggacccagactcggcgcaggg |
| | | | ccaggaacatacccttagaagaactccaaagaaacctccagtttcatgcttttatttcatatagtgaacatga |
| | | | ttctgcctgggtgaaaagtgaattggtaccttacctagaaaaagaagatatacagatttgtcttcatgagaga |
| | | | aactttgtccctggcaagagcattgtggaaaatatcatcaactgcattgagaagagttacaagtccatctttg |
| | | | ttttgtctcccaactttgtccagagtgagtggtgccattacgaactctattttgcccatcacaatctctttcatgaa |
| | | | ggatctaataacttaatcctcatcttactggaacccattccacagaacagcattcccaacaagtaccacaa |
| | | | gctgaaggctctcatgacgcagcggacttatttgcagtggcccaaggagaaaagcaaacgtgggctctttt |
| | | | gggctaacattagagccgcttttaatatgaaattaacactagtcactgaaaacaatgatgtgaaatcttaaa |
| | | | aaaatttaggaaattcaacttaagaaaccattatttacttggatgatggtgaatagtacagtcgtaagtaact |
| | | | gtctggaggtgcctccattatcctcatgccttcaggaaagacttaacaaaaacaatgtttcatctggggaact |
| | | | gagctaggcggtgaggttagcctgccagttagagacagcccagtctcttctggtttaatcattatgtttcaaatt |
| | | | gaaacagtctcttttgagtaaatgctcagtttttcagctcctctccactctgctttcccaaatggattctgttgtga |
| | | | gcaagagtttatatggcttcatggcagcaagggaacagtcaacttcagcatcatatgcaccagtcctcgga |
| | | | gtgccctgtgaatcatattggtctttgggtcagtgtcatcattctcttcaagtctggggcttggggaaaaaatta |
| | | | gatcagctacggcatataaaaaagtcttttgtttcacatatgtgtaatagcttatttaattttttatcctgctacaca |
| | | | aatatgtaattaaccaatgaggactcatgacttgatagtgtatgtatgtaaagggatatatggacttaatcata |
| | | | agctgttgaggtgaaagacgtggatccacctgctttccaagaaaactcggccaaatttatttgcagctggat |
| | | | attgaatgggacttttctggttgtcttagaattctggctaaaggctcaaagctgacgaaagacagtaactgca |
| | | | ccaacatgatactagacacagccagtctggacttatcaaaagagcagaaagagaccaatgactcccag |
| | | | tccgtattatccatctctagaagactagagtcaaaagcgtgattaaagagtcattaagcggaggttctaggc |
| | | | catagggagattgctttgaatttcttgcagacaagtgtgagggactcagcatggtagaaggtagcctggcat |
| | | | cccactccaagactgaaagcttgcagagtaacaggagcacacaggttcagtgcagcagatgtggtgtgg |
| | | | cttgagaattcttggaagagcttgatgagtgtttgctggagtccgaggggggcactgggaacacagagac |
| | | | tggtaaatagtgtttggcaaatacaagtgcttgatgaatatttgttgaatgaatagatgagttcttcccccctgg |
| | | | ggaattcaggaggtgaaaggttggcttgagcacccaaaatggcaggatgagagaagagaagcactga |
| | | | tagcaacctgccctcccattattgacatggtaaaaggatgtgaatttcttcacatggctttgactatggaagag |
| | | | tagctgggcttgcattgtcatgacgggatatcagccaacagggtagcctgttgtgcaaagaaactatagca |
| | | | gtaagaggacacggggttaggcagaagaggggtttggggtggaggttgctgcaagaggtcagccagat |
| | | | aatgtggccctgcatcatggaactgtgcaatgtggggtacactcaaggccctccaataactcacagatgtg |
| | | | ccctatgaaaaagccagcatttggactctgccatagcagctggcaggatcatgctggcctgtctgccttattc |
| | | | aatagttaactacaggaagatctgctcctctttgtgtaataccctcttcccttgcaatggcatagggacatcta |
| | | | gaatatagagaagacagagacaatggaggaagagtaaagaaactgactatatgccttcgtcatttcactg |
| | | | caaggaaggccaagcagatttttgaatgaggtgtgagattgctgttaaattggactggcctggacattttaat |
| | | | cccttaaatagaggtgcaatgactaaagtgagatttgtcactaaaatttatggtatctgcccaagattcagga |
| | | | gtgatgatgggaggagatccaacagaactttgttgtaaggcaatggttagagaaaaatgaagccctcgctt |
| | | | tctggacttagttcattcaataaaccagtttcggccaggcacgttggctcacatctataatcccagtactgtgg |
| | | | gaggctgaggcaggtggatcacttgaggtcaggagttcgagaccagcctggccaacatggtgaaaccct |
| | | | gtctgtactaaaaatacaaaaattagccgggtgtggtggtgtgcacctgtagtcccagctactcgggaggct |
| | | | gaggcaggaaaatcacttgaacctgggagacagaggctgtagtgagctgagacagcgctactgtactcc |
| | | | ccgctgggcaacagagtgagactccatctcaaaaaagttaaaagaaaaaaaatctggtttcataatagct |
| | | | gtaacgaaataagccttaatgatattttattagcatcatcttctgtctgcattagcccttccttgctcttcaggaga |
| | | | acaacatttgttttcctccctaggctctatcccaaacggcacattcttccacaacccctgttgaacagatttttta |
| | | | aactgttgcctaatctaaaaacaataaaaacaacaaacaaccacagtaacaacaacgacaaaaaaaa |
| | | | ctgccacagattctaaataatcagatctttttaaatggtatcaatgtttcccacaaaatattgttgacattgaaa |
| | | | atatagaattttagcattaattttgttaaacctacatcccctcggcagaggggcctccctgcatcccagtgga |
| | | | aagtaggttcctcacagtcctctccgtcacattcttcccatttcttttcttcacagaacacatcactgtctaaaatt |
| | | | atcttgtttgcttagttgcttactcatcttcttcttctctcctctgaagtctaagctccaggaaaaagggagacttct |
| | | | ccacctgttccctgcctctccccagtgccgaggggacactgtgcaccccattgtagatgcgcagtaaaaac |
| | | | tcgtgggatgagcaaatgactctgaaacggtcccatgcgggaaatgtccatgaagtcctggattttatctaa |
| | | | aaagcccaggcaggggggggcgggggcggcggggctacagttccacgctgagctgcctcctggccgc |
| | | | tcgtccccgccgcagtgcctgggcggcccgggcgcccgaccttggccgtggacaccttcgcggtgggtg |
| | | | ctgctcctccccatctgccactggaagatgctggggcgacccggctccaggtttagcaggacactgagaa |
| | | | aagggaatggctgcctttcggaggctgggtgagcccttctctgtgcctcacctgcccgccccacagcggcc |
| | | | ctgcacctcgtcccacggggcccattgccccggtaggatgcgcgcttttgttttgagggtcaggcatcttccct |
| | | | gccgtcgtttctgggaggttgaaaaattgatccagaaagacctaaaacaaaaaacaaagaaaaacaaa |
| | | | aaagcgaaaa |
| Human TLR6 (S249P) | 7 | Amino acid | MTKDKEPIVKSFHFVCLMIIIVGTRIQFSDGNEFAVDKSKRGLIHVPKDLPLKT |
| | | | KVLDMSQNYIAELQVSDMSFLSELTVLRLSHNRIQLLDLSVFKFNQDLEYLD |
| | | | LSHNQLQKISCHPIVSFRHLDLSFNDFKALPICKEFGNLSQLNFLGLSAMKL |
| | | | QKLDLLPIAHLHLSYILLDLRNYYIKENETESLQILNAKTLHLVFHPTSLFAIQV |
| | | | NISVNTLGCLQLTNIKLNDDNCQVFIKFLSELTRGPTLLNFTLNHIETTWKCL |
| | | | VRVFQFLWPKPVEYLNIYNLTIIESIREEDFTYSKTTLKALTIEHITNQVFLFSQ |
| | | | TALYTVFSEMNIMMLTISDTPFIHMLCPHAPSTFKFLNFTQNVFTDSIFEKCS |
| | | | TLVKLETLILQKNGLKDLFKVGLMTKDMPSLEILDVSWNSLESGRHKENCT |
| | | | WVESIVVLNLSSNMLTDSVFRCLPPRIKVLDLHSNKIKSVPKQVVKLEALQE |
| | | | LNVAFNSLTDLPGCGSFSSLSVLIIDHNSVSHPSADFFQSCQKMRSIKAGDN |
| | | | PFQCTCELREFVKNIDQVSSEVLEGWPDSYKCDYPESYRGSPLKDFHMSE |
| | | | LSCNITLLIVTIGATMLVLAVTVTSLCIYLDLPWYLRMVCQWTQTRRRARNIP |
| | | | LEELQRNLQFHAFISYSEHDSAWVKSELVPYLEKEDIQICLHERNFVPGKSI |
| | | | VENIINCIEKSYKSIFVLSPNFVQSEWCHYELYFAHHNLFHEGSNNLILILLEPI |
| | | | PQNSIPNKYHKLKALMTQRTYLQWPKEKSKRGLFWANIRAAFNMKLTLVTE |
| | | | NNDVKS |
| Human TLR6 (S249P) | 8 | Nucleic acid | ccagctcttacctggtagtgtccatcactaagtgtaaagtttggggagaatggagtgtggcagtccgtagaa |
| | | | ggaaaactcagcatgctgatccactcatgtcagtaccattggggtaaaagggacactgcctagctactcat |
| | | | taagtactagttggaactcatcctcactgtttgaatcagagacagagagaggacaaatttctttctgcctttcat |
| | | | cagggagtctattttcctgagacaaagcttgagtgtggcaggtttgccctaacacagttccagttacatttttgt |
| | | | gagtgaagatgctcctggatcctgccattacatttctgtgcttctttgttttcagtggccaagtggatttttcaaatg |
| | | | tatgtgtgtctctgtgggtgttctgatgggcagttgggagaagttatatggatcagtgaagagcttttaaccata |
| | | | cgccattcagtaataaaagttccccaactcagagttttcactcttccaaagaaagaatttggactcatatcaa |
| | | | gatgctctgaagaagaacaaccctttaggatagccactgcaacatcatgaccaaagacaaagaacctat |
| | | | tgttaaaagcttccattttgtttgccttatgatcataatagttggaaccagaatccagttctccgacggaaatga |
| | | | atttgcagtagacaagtcaaaaagaggtcttattcatgttccaaaagacctaccgctgaaaaccaaagtctt |
| | | | agatatgtctcagaactacatcgctgagcttcaggtctctgacatgagctttctatcagagttgacagttttgag |
| | | | actttcccataacagaatccagctacttgatttaagtgttttcaagttcaaccaggatttagaatatttggatttat |
| | | | ctcataatcagttgcaaaagatatcctgccatcctattgtgagtttcaggcatttagatctctcattcaatgatttc |
| | | | aaggccctgcccatctgtaaggaatttggcaacttatcacaactgaatttcttgggattgagtgctatgaagct |
| | | | gcaaaaattagatttgctgccaattgctcacttgcatctaagttatatccttctggatttaagaaattattatataa |
| | | | aagaaaatgagacagaaagtctacaaattctgaatgcaaaaacccttcaccttgtttttcacccaactagttt |
| | | | attcgctatccaagtgaacatatcagttaatactttagggtgcttacaactgactaatattaaattgaatgatga |
| | | | caactgtcaagttttcattaaatttttatcagaactcaccagaggtccaaccttactgaattttaccctcaacca |
| | | | catagaaacgacttggaaatgcctggtcagagtctttcaatttctttggcccaaacctgtggaatatctcaata |
| | | | tttacaatttaacaataattgaaagcattcgtgaagaagattttacttattctaaaacgacattgaaagcattg |
| | | | acaatagaacatatcacgaaccaagtttttctgttttcacagacagctttgtacaccgtgttttctgagatgaac |
| | | | attatgatgttaaccatttcagatacaccttttatacacatgctgtgtcctcatgcaccaagcacattcaagttttt |
| | | | gaactttacccagaacgttttcacagatagtatttttgaaaaatgttccacgttagttaaattggagacacttat |
| | | | cttacaaaagaatggattaaaagaccttttcaaagtaggtctcatgacgaaggatatgccttctttggaaata |
| | | | ctggatgttagctggaattctttggaatctggtagacataaagaaaactgcacttgggttgagagtatagtgg |
| | | | tgttaaatttgtcttcaaatatgcttactgactctgttttcagatgtttacctcccaggatcaaggtacttgatcttca |
| | | | cagcaataaaataaagagcgttcctaaacaagtcgtaaaactggaagctttgcaagaactcaatgttgctt |
| | | | tcaattctttaactgaccttcctggatgtggcagctttagcagcctttctgtattgatcattgatcacaattcagttt |
| | | | cccacccatcggctgatttcttccagagctgccagaagatgaggtcaataaaagcaggggacaatccatt |
| | | | ccaatgtacctgtgagctaagagaatttgtcaaaaatatagaccaagtatcaagtgaagtgttagagggct |
| | | | ggcctgattcttataagtgtgactacccagaaagttatagaggaagcccactaaaggactttcacatgtctg |
| | | | aattatcctgcaacataactctgctgatcgtcaccatcggtgccaccatgctggtgttggctgtgactgtgacc |
| | | | tccctctgcatctacttggatctgccctggtatctcaggatggtgtgccagtggacccagactcggcgcagg |
| | | | gccaggaacatacccttagaagaactccaaagaaacctccagtttcatgcttttatttcatatagtgaacatg |
| | | | attctgcctgggtgaaaagtgaattggtaccttacctagaaaaagaagatatacagatttgtcttcatgagag |
| | | | aaactttgtccctggcaagagcattgtggaaaatatcatcaactgcattgagaagagttacaagtccatcttt |
| | | | gttttgtctcccaactttgtccagagtgagtggtgccattacgaactctattttgcccatcacaatctctttcatga |
| | | | aggatctaataacttaatcctcatcttactggaacccattccacagaacagcattcccaacaagtaccaca |
| | | | agctgaaggctctcatgacgcagcggacttatttgcagtggcccaaggagaaaagcaaacgtgggctctt |
| | | | ttgggctaacattagagccgcttttaatatgaaattaacactagtcactgaaaacaatgatgtgaaatcttaa |
| | | | aaaaatttaggaaattcaacttaagaaaccattatttacttggatgatggtgaatagtacagtcgtaagtaac |
| | | | tgtctggaggtgcctccattatcctcatgccttcaggaaagacttaacaaaaacaatgtttcatctggggaac |
| | | | tgagctaggcggtgaggttagcctgccagttagagacagcccagtctcttctggtttaatcattatgtttcaaat |
| | | | tgaaacagtctcttttgagtaaatgctcagtttttcagctcctctccactctgctttcccaaatggattctgttgtga |
| | | | gcaagagtttatatggcttcatggcagcaagggaacagtcaacttcagcatcatatgcaccagtcctcgga |
| | | | gtgccctgtgaatcatattggtctttgggtcagtgtcatcattctcttcaagtctggggcttggggaaaaaatta |
| | | | gatcagctacggcatataaaaaagtcttttgtttcacatatgtgtaatagcttatttaattttttatcctgctacaca |
| | | | aatatgtaattaaccaatgaggactcatgacttgatagtgtatgtatgtaaagggatatatggacttaatcata |
| | | | agctgttgaggtgaaagacgtggatccacctgctttccaagaaaactcggccaaatttatttgcagctggat |
| | | | attgaatgggacttttctggttgtcttagaattctggctaaaggctcaaagctgacgaaagacagtaactgca |
| | | | ccaacatgatactagacacagccagtctggacttatcaaaagagcagaaagagaccaatgactcccag |
| | | | tccgtattatccatctctagaagactagagtcaaaagcgtgattaaagagtcattaagcggaggttctaggc |
| | | | catagggagattgctttgaatttcttgcagacaagtgtgagggactcagcatggtagaaggtagcctggcat |
| | | | cccactccaagactgaaagcttgcagagtaacaggagcacacaggttcagtgcagcagatgtggtgtgg |
| | | | cttgagaattcttggaagagcttgatgagtgtttgctggagtccgagggtgggcactgggaacacagagac |
| | | | tggtaaatagtgtttggcaaatacaagtgcttgatgaatatttgttgaatgaatagatgagttcttcccccctgg |
| | | | ggaattcaggaggtgaaaggttggcttgagcacccaaaatggcaggatgagagaagagaagcactga |
| | | | tagcaacctgccctcccattattgacatggtaaaaggatgtgaatttcttcacatggctttgactatggaagag |
| | | | tagctgggcttgcattgtcatgacgggatatcagccaacagggtagcctgttgtgcaaagaaactatagca |
| | | | gtaagaggacacggggttaggcagaagaggggtttggggggaggttgctgcaagaggtcagccagat |
| | | | aatgtggccctgcatcatggaactgtgcaatgtggggtacactcaaggccctccaataactcacagatgtg |
| | | | ccctatgaaaaagccagcatttggactctgccatagcagctggcaggatcatgctggcctgtctgccttattc |
| | | | aatagttaactacaggaagatctgctcctctttgtgtaataccctcttcccttgcaatggcatagggacatcta |
| | | | gaatatagagaagacagagacaatggaggaagagtaaagaaactgactatatgccttcgtcatttcactg |
| | | | caaggaaggccaagcagatttttgaatgaggtgtgagattgctgttaaattggactggcctggacattttaat |
| | | | cccttaaatagaggtgcaatgactaaagtgagatttgtcactaaaatttatggtatctgcccaagattcagga |
| | | | gtgatgatgggaggagatccaacagaactttgttgtaaggcaatggttagagaaaaatgaagccctcgctt |
| | | | tctggacttagttcattcaataaaccagtttcggccaggcacgttggctcacatctataatcccagtactgtgg |
| | | | gaggctgaggcaggtggatcacttgaggtcaggagttcgagaccagcctggccaacatggtgaaaccct |
| | | | gtctgtactaaaaatacaaaaattagccgggtgtggtggtgtgcacctgtagtcccagctactcgggaggct |
| | | | gaggcaggaaaatcacttgaacctgggagacagaggctgtagtgagctgagacagcgctactgtactcc |
| | | | ccgctgggcaacagagtgagactccatctcaaaaaagttaaaagaaaaaaaatctggtttcataatagct |
| | | | gtaacgaaataagccttaatgatattttattagcatcatcttctgtctgcattagcccttccttgctcttcaggaga |
| | | | acaacatttgttttcctccctaggctctatcccaaacggcacattcttccacaacccctgttgaacagatttttta |
| | | | aactgttgcctaatctaaaaacaataaaaacaacaaacaaccacagtaacaacaacgacaaaaaaaa |
| | | | ctgccacagattctaaataatcagatctttttaaatggtatcaatgtttcccacaaaatattgttgacattgaaa |
| | | | atatagaattttagcattaattttgttaaacctacatcccctcggcagaggggcctccctgcatcccagtgga |
| | | | aagtaggttcctcacagtcctctccgtcacattcttcccatttcttttcttcacagaacacatcactgtctaaaatt |
| | | | atcttgtttgcttagttgcttactcatcttcttcttctctcctctgaagtctaagctccaggaaaaagggagacttct |
| | | | ccacctgttccctgcctctccccagtgccgaggggacactgtgcaccccattgtagatgcgcagtaaaaac |
| | | | tcgtgggatgagcaaatgactctgaaacggtcccatgcgggaaatgtccatgaagtcctggattttatctaa |
| | | | aaagcccaggcaggggggggcgggggcggggggctacagttccacgctgagctgcctcctggccgc |
| | | | tcgtccccgccgcagtgcctgggcggcccgggcgcccgaccttggccgtggacaccttcgcggtgggtg |
| | | | ctgctcctccccatctgccactggaagatgctggggcgacccggctccaggtttagcaggacactgagaa |
| | | | aagggaatggctgcctttcggaggctgggtgagcccttctctgtgcctcacctgcccgccccacagcggcc |
| | | | ctgcacctcgtcccacggggcccattgccccggtaggatgcgcgcttttgttttgagggtcaggcatcttccct |
| | | | gccgtcgtttctgggaggttgaaaaattgatccagaaagacctaaaacaaaaaacaaagaaaaacaaa |
| | | | aaagcgaaaa |

TABLE 3
TLR10 Alleles.

| Name | SEQ ID NO | Sequence Type | Sequence |
|---|---|---|---|
| Human TLR10 (WT) | 9 | Amino acid | MRLIRNIYIFCSIVMTAEGDAPELPEERELMTNCSNMSLRKVPADLTPAT |
| | | | TTLDLSYNLLFQLQSSDFHSVSKLRVLILCHNRIQQLDLKTFEFNKELRY |
| | | | LDLSNNRLKSVTWYLLAGLRYLDLSFNDFDTMPICEEAGNMSHLEILGLS |
| | | | GAKIQKSDFQKIAHLHLNTVFLGFRTLPHYEEGSLPILNTTKLHIVLPMD |
| | | | TNFWVLLRDGIKTSKILEMTNIDGKSQFVSYEMQRNLSLENAKTSVLLLN |
| | | | KVDLLWDDLFLILQFVWHTSVEHFQIRNVTFGGKAYLDHNSFDYSNTVMR |
| | | | TIKLEHVHFRVFYIQQDKIYLLLTKMDIENLTISNAQMPHMLFPNYPTKF |
| | | | QYLNFANNILTDELFKRTIQLPHLKTLILNGNKLETLSLVSCFANNTPLE |
| | | | HLDLSQNLLQHKNDENCSWPETVVNMNLSYNKLSDSVFRCLPKSIQILDL |
| | | | NNNQIQTVPKETIHLMALRELNIAFNFLTDLPGCSHFSRLSVLNIEMNFI |
| | | | LSPSLDFVQSCQEVKTLNAGRNPFRCTCELKNFIQLETYSEVMMVGWSDS |
| | | | YTCEYPLNLRGTRLKDVHLHELSCNTALLIVTIVVIMLVLGLAVAFCCLH |
| | | | FDLPWYLRMLGQCTQTWHRVRKTTQEQLKRNVRFHAFISYSEHDSLWVK |
| | | | NELIPNLEKEDGSILICLYESYFDPGKSISENIVSFIEKSYKSIFVLSPNF |
| | | | VQNEWCHYEFYFAHHNLFHENSDHIILILLEPIPFYCIPTRYHKLKALLE |
| | | | KKAYLEWPKDRRKCGLFWANLRAAINVNVLATREMYELQTFTELNEESRG |
| | | | STISLMRTDCL |
| Human TLR10 (WT) | 10 | Nucleic acid | agccaattctgaccgtgtcaacgaatcatccacgcacctgcagctctgctgagagagtgcaagccgtggg |
| | | | aattcagcagctgaatatcaagacctttgaattcaacaagaagttaagacatttatagttgtctaacaacag |
| | | | actgaagattgtggcttggtattcactggcaggtttcagacatttagatctttcttttaatgactaacaccatgcct |
| | | | atctgtggagaagctggcaacatgtcacacctggaaattgtttttcaacattaatactattatttggcagtaatc |
| | | | cagattgcttttgccaccaacctgaagacatatagaggcagaaggacaggaataattctatttgtttcctgttt |
| | | | tgaaacttccatctgtaaggctatcaaaaggagatgtgagagagggtattgagtctggcctgacaatgcag |
| | | | ttcttaaaccaaaggtccattatgcttctcctctctgagaatcctgacttacctcaacaacggagacatggca |
| | | | cagtagccagcttggagacttctcagccaatgctctgagatcaagtcgaagacccaatatacagggttttg |
| | | | agctcatcttcatcattcatatgaggaaataagtggtaaaatccttggaaatacaatgagactcatcagaaa |
| | | | catttacatattttgtagtattgttatgacagcagagggtgatgctccagagctgccagaagaaagggaact |
| | | | gatgaccaactgctccaacatgtctctaagaaaggttcccgcagacttgaccccagccacaacgacact |
| | | | ggatttatcctataacctcctttttcaactccagagttcagattttcattctgtctccaaactgagagttttgattcta |
| | | | tgccataacagaattcaacagctggatctcaaaacctttgaattcaacaaggagttaagatatttagatttgt |
| | | | ctaataacagactgaagagtgtaacttggtatttactggcaggtctcaggtatttagatctttcttttaatgacttt |
| | | | gacaccatgcctatctgtgaggaagctggcaacatgtcacacctggaaatcctaggtttgagtggggcaa |
| | | | aaatacaaaaatcagatttccagaaaattgctcatctgcatctaaatactgtcttcttaggattcagaactcttc |
| | | | ctcattatgaagaaggtagcctgcccatcttaaacacaacaaaactgcacattgttttaccaatggacaca |
| | | | aatttctgggttcttttgcgtgatggaatcaagacttcaaaaatattagaaatgacaaatatagatggcaaaa |
| | | | gccaatttgtaagttatgaaatgcaacgaaatcttagtttagaaaatgctaagacatcggttctattgcttaata |
| | | | aagttgatttactctgggacgaccttttccttatcttacaatttgtttggcatacatcagtggaacactttcagatc |
| | | | cgaaatgtgacttttggtggtaaggcttatcttgaccacaattcatttgactactcaaatactgtaatgagaact |
| | | | ataaaattggagcatgtacatttcagagtgttttacattcaacaggataaaatctatttgcttttgaccaaaatg |
| | | | gacatagaaaacctgacaatatcaaatgcacaaatgccacacatgcttttcccgaattatcctacgaaatt |
| | | | ccaatatttaaattttgccaataatatcttaacagacgagttgtttaaaagaactatccaactgcctcacttgaa |
| | | | aactctcattttgaatggcaataaactggagacactttctttagtaagttgctttgctaacaacacacccttgga |
| | | | acacttggatctgagtcaaaatctattacaacataaaaatgatgaaaattgctcatggccagaaactgtggt |
| | | | caatatgaatctgtcatacaataaattgtctgattctgtcttcaggtgcttgcccaaaagtattcaaatacttgac |
| | | | ctaaataataaccaaatccaaactgtacctaaagagactattcatctgatggccttacgagaactaaatatt |
| | | | gcatttaattttctaactgatctccctggatgcagtcatttcagtagactttcagttctgaacattgaaatgaactt |
| | | | cattctcagcccatctctggattttgttcagagctgccaggaagttaaaactctaaatgcgggaagaaatcc |
| | | | attccggtgtacctgtgaattaaaaaatttcattcagcttgaaacatattcagaggtcatgatggttggatggtc |
| | | | agattcatacacctgtgaataccctttaaacctaaggggaactaggttaaaagacgttcatctccacgaatt |
| | | | atcttgcaacacagctctgttgattgtcaccattgtggttattatgctagttctggggttggctgtggccttctgctg |
| | | | tctccactttgatctgccctggtatctcaggatgctaggtcaatgcacacaaacatggcacagggttaggaa |
| | | | aacaacccaagaacaactcaagagaaatgtccgattccacgcatttatttcatacagtgaacatgattctct |
| | | | gtgggtgaagaatgaattgatccccaatctagagaaggaagatggttctatcttgatttgcctttatgaaagct |
| | | | actttgaccctggcaaaagcattagtgaaaatattgtaagcttcattgagaaaagctataagtccatctttgttt |
| | | | tgtctcccaactttgtccagaatgagtggtgccattatgaattctactttgcccaccacaatctcttccatgaaa |
| | | | attctgatcatataattcttatcttactggaacccattccattctattgcattcccaccaggtatcataaactgaa |
| | | | agctctcctggaaaaaaaagcatacttggaatggcccaaggataggcgtaaatgtgggcttttctgggca |
| | | | aaccttcgagctgctattaatgttaatgtattagccaccagagaaatgtatgaactgcagacattcacagag |
| | | | ttaaatgaagagtctcgaggttctacaatctctctgatgagaacagattgtctataaaatcccacagtccttgg |
| | | | gaagttggggaccacatacactgttgggatgtacattgatacaacctttatgatggcaatttgacaatatttatt |
| | | | aaaataaaaaatggttattcccttcatatcagtttctagaaggatttctaagaatgtatcctatagaaacacctt |
| | | | cacaagtttataagggcttatggaaaaaggtgttcatcccaggattgtttataatcatgaaaaatgtggccag |
| | | | gtgcagtggctcactcttgtaatcccagcactatgggaggccaaggtgggtgacccacgaggtcaagag |
| | | | atggagaccatcctggccaacatggtgaaaccctgtctctactaaaaatacaaaaattagctgggcgtgat |
| | | | ggtgcacgcctgtagtcccagctacttgggaggctgaggcaggagaatcgcttgaacccgggaggtggc |
| | | | agttgcagtgagctgagatcgagccactgcactccagcctggtgacagagcgagactccatctcaaaaa |
| | | | aaagaaaaaaaaaaaagaaaaaaatggaaaacatcctcatggccacaaaataaggtctaattcaata |
| | | | aattatagtacattaatgtaatataatattacatgccactaaaaagaataaggtagctgtatatttcctggtatg |
| | | | gaaaaaacatattaatatgttataaactattaggttggtgcaaaactaattgtggtttttgccattgaaatggca |
| | | | ttgaaataaaagtgtaaagaaatctataccagatgtagtaacagtggtttgggtctgggaggttggattaca |
| | | | gggagcatttgatttctatgttgtgtatttctataatgtttgaattgtttagaatgaatctgtatttcttttataagtaga |
| | | | aaaaaaataaagatagtttttacagccta |
| Human TLR10 (I775V) | 11 | Amino acid | MRLIRNIYIFCSIVMTAEGDAPELPEERELMTNCSNMSLRKVPADLTPAT |
| | | | TTLDLSYNLLFQLQSSDFHSVSKLRVLILCHNRIQQLDLKTFEFNKELRY |
| | | | LDLSNNRLKSVTWYLLAGLRYLDLSFNDFDTMPICEEAGNMSHLEILGLS |
| | | | GAKIQKSDFQKIAHLHLNTVFLGFRTLPHYEEGSLPILNTTKLHIVLPMD |
| | | | TNFWVLLRDGIKTSKILEMTNIDGKSQFVSYEMQRNLSLENAKTSVLLLN |
| | | | KVDLLWDDLFLILQFVWHTSVEHFQIRNVTFGGKAYLDHNSFDYSNTVMR |
| | | | TIKLEHVHFRVFYIQQDKIYLLLTKMDIENLTISNAQMPHMLFPNYPTKF |
| | | | QYLNFANNILTDELFKRTIQLPHLKTLILNGNKLETLSLVSCFANNTPLE |
| | | | HLDLSQNLLQHKNDENCSWPETVVNMNLSYNKLSDSVFRCLPKSIQILDL |
| | | | NNNQIQTVPKETIHLMALRELNIAFNFLTDLPGCSHFSRLSVLNIEMNFI |
| | | | LSPSLDFVQSCQEVKTLNAGRNPFRCTCELKNFIQLETYSEVMMVGWSDS |
| | | | YTCEYPLNLRGTRLKDVHLHELSCNTALLIVTIVVIMLVLGLAVAFCCLH |
| | | | FDLPWYLRMLGQCTQTWHRVRKTTQEQLKRNVRFHAFISYSEHDSLWVK |
| | | | NELIPNLEKEDGSILICLYESYFDPGKSISENIVSFIEKSYKSIFVLSPNF |
| | | | VQNEWCHYEFYFAHHNLFHENSDHIILILLEPIPFYCIPTRYHKLKALLE |
| | | | KKAYLEWPKDRRKCGLFWANLRAAVNVNVLATREMYELQTFTELNEESRG |
| | | | STISLMRTDCL |
| Human TLR10 (I775V) | 12 | Nucleic acid | agccaattctgaccgtgtcaacgaatcatccacgcacctgcagctctgctgagagagtgcaagccgtggg |
| | | | aattcagcagctgaatatcaagacctttgaattcaacaagaagttaagacatttatagttgtctaacaacag |
| | | | actgaagattgtggcttggtattcactggcaggtttcagacatttagatctttcttttaatgactaacaccatgcct |
| | | | atctgtggagaagctggcaacatgtcacacctggaaattgtttttcaacattaatactattatttggcagtaatc |
| | | | cagattgcttttgccaccaacctgaagacatatagaggcagaaggacaggaataattctatttgtttcctgttt |
| | | | tgaaacttccatctgtaaggctatcaaaaggagatgtgagagagggtattgagtctggcctgacaatgcag |
| | | | ttcttaaaccaaaggtccattatgcttctcctctctgagaatcctgacttacctcaacaacggagacatggca |
| | | | cagtagccagcttggagacttctcagccaatgctctgagatcaagtcgaagacccaatatacagggttttg |
| | | | agctcatcttcatcattcatatgaggaaataagtggtaaaatccttggaaatacaatgagactcatcagaaa |
| | | | catttacatattttgtagtattgttatgacagcagagggtgatgctccagagctgccagaagaaagggaact |
| | | | gatgaccaactgctccaacatgtctctaagaaaggttcccgcagacttgaccccagccacaacgacact |
| | | | ggatttatcctataacctcctttttcaactccagagttcagattttcattctgtctccaaactgagagttttgattcta |
| | | | tgccataacagaattcaacagctggatctcaaaacctttgaattcaacaaggagttaagatatttagatttgt |
| | | | ctaataacagactgaagagtgtaacttggtatttactggcaggtctcaggtatttagatctttcttttaatgacttt |
| | | | gacaccatgcctatctgtgaggaagctggcaacatgtcacacctggaaatcctaggtttgagtggggcaa |
| | | | aaatacaaaaatcagatttccagaaaattgctcatctgcatctaaatactgtcttcttaggattcagaactcttc |
| | | | ctcattatgaagaaggtagcctgcccatcttaaacacaacaaaactgcacattgttttaccaatggacaca |
| | | | aatttctgggttcttttgcgtgatggaatcaagacttcaaaaatattagaaatgacaaatatagatggcaaaa |
| | | | gccaatttgtaagttatgaaatgcaacgaaatcttagtttagaaaatgctaagacatcggttctattgcttaata |
| | | | aagttgatttactctgggacgaccttttccttatcttacaatttgtttggcatacatcagtggaacactttcagatc |
| | | | cgaaatgtgacttttggtggtaaggcttatcttgaccacaattcatttgactactcaaatactgtaatgagaact |
| | | | ataaaattggagcatgtacatttcagagtgttttacattcaacaggataaaatctatttgcttttgaccaaaatg |
| | | | gacatagaaaacctgacaatatcaaatgcacaaatgccacacatgcttttcccgaattatcctacgaaatt |
| | | | ccaatatttaaattttgccaataatatcttaacagacgagttgtttaaaagaactatccaactgcctcacttgaa |
| | | | aactctcattttgaatggcaataaactggagacactttctttagtaagttgctttgctaacaacacacccttgga |
| | | | acacttggatctgagtcaaaatctattacaacataaaaatgatgaaaattgctcatggccagaaactgtggt |
| | | | caatatgaatctgtcatacaataaattgtctgattctgtcttcaggtgcttgcccaaaagtattcaaatacttgac |
| | | | ctaaataataaccaaatccaaactgtacctaaagagactattcatctgatggccttacgagaactaaatatt |
| | | | gcatttaattttctaactgatctccctggatgcagtcatttcagtagactttcagttctgaacattgaaatgaactt |
| | | | cattctcagcccatctctggattttgttcagagctgccaggaagttaaaactctaaatgcgggaagaaatcc |
| | | | attccggtgtacctgtgaattaaaaaatttcattcagcttgaaacatattcagaggtcatgatggttggatggtc |
| | | | agattcatacacctgtgaataccctttaaacctaaggggaactaggttaaaagacgttcatctccacgaatt |
| | | | atcttgcaacacagctctgttgattgtcaccattgtggttattatgctagttctggggttggctgtggccttctgctg |
| | | | tctccactttgatctgccctggtatctcaggatgctaggtcaatgcacacaaacatggcacagggttaggaa |
| | | | aacaacccaagaacaactcaagagaaatgtccgattccacgcatttatttcatacagtgaacatgattctct |
| | | | gtgggtgaagaatgaattgatccccaatctagagaaggaagatggttctatcttgatttgcctttatgaaagct |
| | | | actttgaccctggcaaaagcattagtgaaaatattgtaagcttcattgagaaaagctataagtccatctttgttt |
| | | | tgtctcccaactttgtccagaatgagtggtgccattatgaattctactttgcccaccacaatctcttccatgaaa |
| | | | attctgatcatataattcttatcttactggaacccattccattctattgcattcccaccaggtatcataaactgaa |
| | | | agctctcctggaaaaaaaagcatacttggaatggcccaaggataggcgtaaatgtgggcttttctgggca |
| | | | aaccttcgagctgctgttaatgttaatgtattagccaccagagaaatgtatgaactgcagacattcacagag |
| | | | ttaaatgaagagtctcgaggttctacaatctctctgatgagaacagattgtctataaaatcccacagtccttgg |
| | | | gaagttggggaccacatacactgttgggatgtacattgatacaacctttatgatggcaatttgacaatatttatt |
| | | | aaaataaaaaatggttattcccttcatatcagtttctagaaggatttctaagaatgtatcctatagaaacacctt |
| | | | cacaagtttataagggcttatggaaaaaggtgttcatcccaggattgtttataatcatgaaaaatgtggccag |
| | | | gtgcagtggctcactcttgtaatcccagcactatgggaggccaaggtgggtgacccacgaggtcaagag |
| | | | atggagaccatcctggccaacatggtgaaaccctgtctctactaaaaatacaaaaattagctgggcgtgat |
| | | | ggtgcacgcctgtagtcccagctacttgggaggctgaggcaggagaatcgcttgaacccgggaggtggc |
| | | | agttgcagtgagctgagatcgagccactgcactccagcctggtgacagagcgagactccatctcaaaaa |
| | | | aaagaaaaaaaaaaaagaaaaaaatggaaaacatcctcatggccacaaaataaggtctaattcaata |
| | | | aattatagtacattaatgtaatataatattacatgccactaaaaagaataaggtagctgtatatttcctggtatg |
| | | | gaaaaaacatattaatatgttataaactattaggttggtgcaaaactaattgtggtttttgccattgaaatggca |
| | | | ttgaaataaaagtgtaaagaaatctataccagatgtagtaacagtggtttgggtctgggaggttggattaca |
| | | | gggagcatttgatttctatgttgtgtatttctataatgtttgaattgtttagaatgaatctgtatttottttataagtaga |
| | | | aaaaaaataaagatagtttttacagccta |

The terms “subject” and “patient” may be used interchangeably herein to refer to any animal, such as any mammal, including but not limited to, humans, non-human primates, rodents, and the like. In some embodiments, the subject or patient is a mammal. In some embodiments, the subject or patient is a human.

As used herein, “intratumoral administration” and “intra-tumoral injection” are used interchangeably to mean delivery of an agent directly to a tumor tissue.

As used herein, “long-axis diameter” means the length of the tumor as measured on the longest axis, e.g., according to modified response evaluation criteria in solid tumors (RECIST) 1.1 using CT/MRI and/or photography. In some embodiments, a STING agonist is administered to a patient with a solid tumor, wherein the long-axis diameter of the solid tumor is greater than or equal to 1 cm. In some embodiments, a STING agonist is administered to a patient with a lymphoma, wherein the long-axis diameter of the lymphoma is greater than or equal to 1 cm.

As used herein, “short-axis diameter” means the length of the tumor, e.g., as measured on the short axis according to modified Response evaluation criteria in solid tumors (RECIST) 1.1 using CT/MRI and/or photography. In some embodiments, a STING agonist is administered to a patient with a lymph node lesion, wherein the short-axis diameter of the lymph node is greater than or equal to 1.5 cm.

Administration “in combination” or “co-administration,” as used herein, means that two or more different treatments are delivered to a patient during the patient's affliction with a disease or disorder (e.g., a cancer). For example, in some embodiments, the two or more treatments are delivered after the patient has been diagnosed with a disease or disorder, and before the disease or disorder has been cured or eliminated. In some embodiments, the delivery of one treatment is still occurring when the delivery of the second treatment begins, so that there is overlap. In some embodiments, the first and second treatment are initiated at the same time. These types of delivery are sometimes referred to herein as “simultaneous,” “concurrent,” or “concomitant” delivery. In other embodiments, the delivery of at least one dose of one treatment ends before delivery of a dose of the second treatment begins. This type of delivery is sometimes referred to herein as “successive” or “sequential” delivery. In some embodiments, a STING agonist is administered to a patient alone or in combination with at least one additional therapy. In some embodiments of combination treatment, the at least one additional therapy comprises administering a checkpoint inhibitor. In some embodiments, a STING agonist and a checkpoint inhibitor are administered simultaneously. In some embodiments, a STING agonist and a checkpoint inhibitor are administered sequentially. In either case, the two treatments should be administered sufficiently close in time so as to provide the desired therapeutic effect.

As used herein, the term “checkpoint inhibitor” refers to any therapeutic agent, including any small molecule chemical compound, antibody, nucleic acid molecule, or polypeptide, or any fragments thereof, or any conjugate thereof, that inhibits one or more of the immune checkpoint pathways, thereby allowing more extensive immune activity. Immune checkpoints are signaling pathways that limit the activity of the immune response. Inhibition of an immune checkpoint pathway may result in an increased immune response. In some embodiments, a checkpoint inhibitor is targeted at PD1, PDL1, and/or CTLA4.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, such as histological examination. The definition of a cancer cell, as used herein, includes not only a primary cancer cell, but also any cell derived from a cancer cell ancestor. This includes metastasized cancer cells, and in vitro cultures and cell lines derived from cancer cells. A cancer may manifest as a solid tumor, e.g., a tumor detectable on the basis of tumor mass, e.g., by procedures such as computed tomography (CT) scan, magnetic resonance imaging (MRI), X-ray, ultrasound or palpation on physical examination, and/or be detectable because of the expression of one or more cancer-specific antigens in a sample obtainable from a patient. A cancer may be a hematological (or hematopoietic or blood-related) malignancy, for example, a cancer derived from blood cells or immune cells, which may be referred to as a liquid tumor. Specific examples of hematological malignancies include leukemias; plasma cell malignancies; lymphomas; and the like. In some embodiments, a human subject has been or concurrently is diagnosed with cancer prior to treatment. In some embodiments, a cancer described herein may be any solid tumor and/or hematological malignancy. In some embodiments, the cancer comprises a cancer of the upper aerodigestive tract. In some embodiments, the cancer comprises a cancer of the lip, buccal mucosa, floor of mouth, oral cavity, hard palate, base of tongue, oral tongue, tonsil, oropharynx, salivary gland, gallbladder, esophagus, stomach, and/or biliary tract. In some embodiments, the cancer comprises a head and neck squamous cell carcinoma (e.g., a cancer of the lip, buccal mucosa, floor of mouth, oral cavity, hard palate, base of tongue, oral tongue, tonsil, and/or oropharynx). In some embodiments, the cancer comprises an esophageal cancer. In some embodiments, the cancer is a recurrent cancer. In some embodiments, the cancer is a metastatic cancer.

As used herein, “cancer of the upper aerodigestive tract” comprises cancer of the organs and tissues of the respiratory tract and the upper part of the digestive tract. In some embodiments, cancer of the upper aerodigestive tract refers to cancer of the lip. In some embodiments, cancer of the upper aerodigestive tract comprises cancer of the mouth. In some embodiments, cancer of the upper aerodigestive tract comprises cancer of the tongue. In some embodiments, cancer of the upper aerodigestive tract comprises cancer of the nose. In some embodiments, cancer of the upper aerodigestive tract comprises cancer of the throat. In some embodiments, cancer of the upper aerodigestive tract comprises cancer of the vocal cords. In some embodiments, cancer of the upper aerodigestive tract comprises cancer of the esophagus. In some embodiments, cancer of the upper aerodigestive tract comprises cancer of the windpipe. In some embodiments, cancer of the upper aerodigestive tract comprises gastric cancer. In some embodiments, cancer of the upper aerodigestive tract comprises cancer of the biliary track.

As used herein, “head and neck squamous cell carcinoma” or “HNSCC” comprises cancer of the lip, buccal mucosa, floor of mouth, oral cavity, hard palate, base of tongue, oral tongue, tonsil, and/or oropharynx.

In some embodiments, a “recurrent” cancer is a cancer that has returned after a period during which the cancer could not be detected.

In some embodiments, “metastatic” cancer is a cancer that has spread from the primary site to another place in the human body.

### Therapeutic Methods and Uses

In various embodiments, the methods and uses described herein comprise obtaining a biological sample from the patient and determining from the sample whether the patient's genotype includes single nucleotide variant(s) rs7447927, rs5743810, and/or rs4129009. In some embodiments, the biological sample comprises a buccal sample, a blood sample, and/or a tumor sample. In some embodiments, the biological sample is obtained from the patient by a buccal swab, a phlebotomy, and/or a tumor biopsy. In some embodiments, treatment decisions, e.g., whether to administer a STING agonist such as E7766 or a pharmaceutically acceptable salt thereof, are based on the determination of whether single nucleotide variant(s) rs7447927, rs5743810, and/or rs4129009 are present in the sample. In some embodiments, a patient is administered a treatment comprising a STING agonist (e.g., alone or in combination with at least one additional agent such as a checkpoint inhibitor) based on genotyping from the sample.

### Single Nucleotide Variants, Methods of Detection, and Patient Selection

In some embodiments, the methods and uses described herein comprise obtaining a biological sample from the patient and determining from the sample whether the patient's genotypes in TMEM173 and TLR6, the combination of genotypes in TMEM173 and TLR10, or in all three genes, in order to determine treatment decisions, e.g., with a STING agonist. In some embodiments, evaluating the genotype comprises checking for single nucleotide variant(s) rs7447927, rs5743810, and/or rs4129009. In some embodiments, the biological sample comprises a buccal sample, a blood sample, and/or a tumor sample. In some embodiments, the biological sample is obtained from the patient by a buccal swab, a phlebotomy, and/or a tumor biopsy. Such samples can be obtained according to methods known to those skilled in the art.

In some embodiments, the patient's genotype for single nucleotide variant(s) rs7447927, rs5743810, and/or rs4129009 is determined by DNA variant testing. In some embodiments, the DNA variant testing comprises next generation sequencing (NGS), polymerase chain reaction (PCR), and/or pyrosequencing.

In some embodiments, evaluating genotypes in a patient sample comprises determining whether the sample is reference (i.e., wild-type or standard on both alleles), variant (non-wild type on both alleles), or heterozygous (one wild-type allele and one variant allele) for TMEM173. In some embodiments, evaluating genotypes in a patient sample comprises determining whether the sample is wild type, variant, or heterozygous for TLR6. In some embodiments, evaluating genotypes in a patient sample comprises determining whether the sample is wild type, variant, or heterozygous for TLR10. In some embodiments, evaluating the sample comprises determining the presence or absence of a rs7447927 genotype for TMEM173 and the presence or absence of a rs5743810 genotype for TLR6. In some embodiments, evaluating the sample comprises determining the presence or absence of a rs7447927 genotype for TMEM173 and the presence or absence of a rs4129009 genotype for TLR10. In some embodiments, evaluating the sample comprises determining the presence or absence of a rs7447927 genotype for TMEM173, the presence or absence of a rs5743810 genotype for TLR6, and the presence or absence of a rs4129009 genotype for TLR10. In some embodiments, evaluating the sample determining the presence or absence of a rs7447927 genotype for TMEM173 and the presence or absence of a rs5743810 genotype for TLR6. In some embodiments, evaluating the sample comprises determining the presence or absence of a rs7447927 genotype for TMEM173 and the presence or absence of a rs4129009 genotype for TLR10. In some embodiments, evaluating the sample comprises determining the presence or absence of a rs7447927 genotype for TMEM173, the presence or absence of a rs5743810 genotype TLR6, and the presence or absence of a rs4129009 genotype for TLR10.

In some embodiments, the wild-type TMEM173 allele comprises or consists of SEQ ID NO: 2. In some embodiments, the allelic variant of human TMEM173 gene (TMEM173 V48V) comprises or consists of any sequence other than SEQ ID NO: 2, e.g., SEQ ID NOs: 3 or 4. In some embodiments, the wild-type TLR6 allele comprises or consists of SEQ ID NO: 6. In some embodiments, the allelic variant of human TLR6 gene (TLR6 S249P) comprises or consists of any sequence other than SEQ ID NO: 6, e.g., SEQ ID NO: 8. In some embodiments, the wild-type TLR10 allele comprises or consists of SEQ ID NO: 10. In some embodiments, the allelic variant of human TLR10 gene (TLR10 I775V) comprises or consists of any sequence other than SEQ ID NO: 10, e.g., SEQ ID NO: 12. In some embodiments, the variants comprise any of those noted below in Table 4.

TABLE 4
Single Nucleotide Variants for TMEM173, TLR6, TLR10

| Gene (Reference SNP) | Reference | Variant |
|---|---|---|
| TMEM173 (rs7447927) | C | G, T |
| TLR6 (rs5743810) | T | C |
| TLR10 (rs4129009) | A | G |

TABLE 5
TMEM173, TLR6, TLR10 Zygosity -
Exemplary Treatment Criteria

| TMEM173 V48V | TLR6 S249P | Candidate for |
|---|---|---|
| (rs7447927) | (rs5743810) | Treatment? |
| reference | reference | yes |
| reference | heterozygous | yes |
| reference | homozygous | no |
| heterozygous | reference | yes |
| heterozygous | heterozygous | yes |
| heterozygous | homozygous | no |
| homozygous | reference | no |
| homozygous | heterozygous | no |
| homozygous | homozygous | no |
| TMEM173 V48V | TLR10 I775V | Candidate for |
| (rs7447927) | (rs4129009) | Treatment? |
| reference | reference | yes |
| reference | heterozygous | no |
| reference | homozygous | no |
| heterozygous | reference | yes |
| heterozygous | heterozygous | no |
| heterozygous | homozygous | no |
| homozygous | reference | no |
| homozygous | heterozygous | no |
| homozygous | homozygous | no |

In some embodiments, a patient is a candidate for treatment based on the patient's genotype for single nucleotide variant(s) rs7447927, rs5743810, and/or rs4129009, as described in Table 5. In some embodiments, the patient is reference for rs7447927 and reference for rs5743810. In some embodiments, the patient is reference for rs7447927 and heterozygous for rs5743810. In some embodiments, the patient is heterozygous for rs7447927 and reference for rs5743810. In some embodiments, the patient is heterozygous for rs7447927 and heterozygous for rs5743810. In some embodiments, the patient is reference for rs7447927 and reference for rs4129009. In some embodiments, the patient is heterozygous for rs7447927 and reference for rs4129009.

In some embodiments, the present disclosure provides methods of treating a cancer in a patient, comprising: (a) determining that the patient carries: (i) reference or heterozygous DNA sequences for TMEM173 V48V (rs7447927); and (ii) reference or heterozygous DNA sequences for TLR6 S249P (rs5743810); or reference DNA sequences for TLR10 I1775V (rs4129009); and (b) administering a therapeutically effective amount of a STING agonist to the patient. In some embodiments, the present disclosure provides methods of treating a cancer in a patient, comprising administering a therapeutically effective amount of a STING agonist to the patient who carries: (i) reference or heterozygous DNA sequences for TMEM173 V48V (rs7447927); and (ii) reference or heterozygous DNA sequences for TLR6 S249P (rs5743810); or reference DNA sequences for TLR10 I1775V (rs4129009). In some embodiments, the present disclosure provides use of a patient's genotype for single nucleotide variant(s) rs7447927, rs5743810, and/or rs4129009 as a biomarker in the manufacture of a medicament for treating a cancer in the patient. In some embodiments, the present disclosure provides use of a patient's genotype for single nucleotide variant(s) rs7447927, rs5743810, and/or rs4129009 as a biomarker in the manufacture of a medicament for treating a cancer in the patient. In some embodiments, the present disclosure provides use of a patient's genotype for single nucleotide variant(s) rs7447927, rs5743810, and/or rs4129009 as a biomarker for treating a cancer in the patient. In some embodiments, the treating comprises: (a) determining that the patient carries: (i) reference or heterozygous DNA sequences for TMEM173 V48V (rs7447927); and (ii) reference or heterozygous DNA sequences for TLR6 S249P (rs5743810); or reference DNA sequences for TLR10 I775V (rs4129009); and (b) administering a therapeutically effective amount of a STING agonist to the patient.

In various embodiments, the STING agonist may be administered in a pharmaceutical composition. The pharmaceutical composition may comprise a second agent, e.g., at least one checkpoint inhibitor. In some embodiments, the at least one checkpoint inhibitor is in a second pharmaceutical composition that is administered simultaneously or sequentially with the STING agonist or pharmaceutical composition comprising the STING agonist. In some embodiments, the at least one checkpoint inhibitor is in a second pharmaceutical composition that is administered separately (e.g., at a separate clinical visit) from the STING agonist or pharmaceutical composition comprising the STING agonist

### Therapeutic Compounds

In various embodiments, the methods disclosed herein comprise administering a STING agonist or pharmaceutical composition comprising a STING agonist to a patient in need thereof, e.g., one identified as suitable for treatment with a STING agonist according to the methods disclosed herein. In some embodiments, the STING agonist comprises one or more of E7766 or a pharmaceutically acceptable salt thereof, ADU-S100, BI 1387446, MK-1454, MK-2118, BMS-986301, CDK-002, GSK-3745417, idronoxil, SB-11285, IMSA-101, SNX-281, SYNB-1891, TAK-676, DMXAA, FAA, CMA, α-Mangostin, BNBC, DSDP, diABZI, a bicyclic benzamide, a benzothiophene, MSA-2, SR-717, MAVU-104, TTI-10001, SRCB-0001, CRD 5500, ALG-031048, JNJ-′6196, IACS-8803, IACS-8779, NZ-IO-STING, OX-401, SITX-799, STACT-TREX1, XMT-2056, ONM-500, MV-626, PF-06928215, DN-15089, HH18202, and/or STI-001. In some embodiments, the STING agonist comprises one or more of E7766 or a pharmaceutically acceptable salt thereof, ADU-S100, BI 1387446, MK-1454, MK-2118, BMS-986301, CDK-002, GSK-3745417, idronoxil, SB-11285, IMSA-101, SNX-281, SYNB-1891, and/or TAK-676. In some embodiments, the STING agonist comprises E7766 or a pharmaceutically acceptable salt thereof, such as its diammonium salt.

In some embodiments, the methods disclosed herein comprise administering the STING agonist or pharmaceutical composition comprising a STING agonist in conjunction with at least one additional agent, such as a checkpoint inhibitor or pharmaceutical composition comprising a checkpoint inhibitor, to the patient in need thereof. In some embodiments, a checkpoint inhibitor is an inhibitor of the programmed death-1 (PD1) pathway. The programmed cell death 1 (PD1) pathway represents an immune control switch which may be engaged by tumor cells to overcome active T-cell immune surveillance. The ligands for the PD1 receptor (PDL1 and PDL2) are constitutively expressed or can be induced in various tumors. As used herein, the term “PD1 inhibitor” refers to any inhibitor of PD1 and/or the PD1 pathway unless the context indicates that it refers specifically to an inhibitor that acts directly on the PD1 receptor. Exemplary PD1 inhibitors include but are not limited to anti-PD1 and anti-PDL1 antibodies.

In some embodiments, the checkpoint inhibitor is an anti-PD1 antibody that binds to the PD-1 receptor. Exemplary anti-PD1 antibodies include but are not limited to pembrolizumab (MK-3475), nivolumab, and/or spartalizumab.

In some embodiments, the checkpoint inhibitor is an anti-PDL1 antibody. Exemplary anti-PDL1 antibodies include but are not limited to atezolizumab, avelumab, and durvalumab.

In other embodiments, a checkpoint inhibitor is an inhibitor of the cytotoxic T-lymphocyte-associated antigen (CTLA4) pathway. CTLA4, also known as CD152, is a protein receptor that downregulates immune responses. CTLA4 is constitutively expressed in regulatory T-cells, but only upregulated in conventional T-cells after activation.

As used herein, the term “CTLA4 inhibitor” refers to any inhibitor of CTLA4 and/or the CTLA4 pathway unless the context indicates that it refers specifically to an inhibitor that acts directly on CTLA4. Exemplary CTLA4 inhibitors include but are not limited to anti-CTLA4 antibodies. In some embodiments, the CTLA4 inhibitor comprises an anti-CTLA4 antibody.

In some embodiments, a checkpoint inhibitor is targeted at PD1/PDL1, CTLA4, OX40, CD40, LAG3, TIM3, GITR, and/or KIR (including a multispecific antibody that may bind to more than one epitope on one or more checkpoint antigen targets). In some embodiments, a checkpoint inhibitor is targeted at CTLA4, OX40, CD40, and/or GITR. In some embodiments, a checkpoint inhibitor is targeted by administering an inhibitory antibody or other similar inhibitory molecule (e.g., an inhibitory anti-CTLA4 or anti-PD1/PDL1 antibody). In some embodiments, a checkpoint inhibitor is targeted by administering an agonist for the target; examples of targets in this class include the stimulatory targets OX40, CD40, and/or GITR. In some embodiments, the checkpoint inhibitor is an agonist antibody for OX40, CD40, and/or GITR. Agonist antibodies directed against OX40 may have a dual role, inhibiting regulatory T-cell suppression, while enhancing effector T-cell functions. Agonist anti-GITR antibodies have also been shown to make effector T-cells more resistant to the inhibition induced by regulatory T-cells (Karaki et al. (2016) Vaccines (Basel) 4 (4): 37). Likewise, agonist CD40 antibodies demonstrate T-cell-dependent anti-tumor activity. Activation of CD40 on dendritic cells increases cross-presentation of tumor antigens and consequently the number of activated tumor-directed effector T-cells (Ellmark et al. (2015) Oncoimmunol. 4 (7): e1011484).

### Treatment Regimens

By virtue of its agonist activity, a STING agonist may be useful in treating cancer in a subject in need thereof, including various types of cancerous growths, oncogenic processes, metastatic tissues, or malignantly transformed cells, tissues, or organs. The inventors have discovered that STING agonists may be particularly effective in treating cancer in patients exhibiting particular genotypes, e.g. those discussed above.

As used herein, a patient is “suitable for” or “in need of” a treatment if such patient would benefit biologically, medically, and/or in quality of life from such treatment. In some embodiments, a patient suitable for treatment with a STING agonist (e.g., E7766 or a pharmaceutically acceptable salt thereof) is a cancer patient who has a particular genotype comprising combinations of single nucleotide variant(s) rs7447927, rs5743810, and/or rs4129009. In some embodiments, the patient's genotype for single nucleotide variant(s) rs7447927, rs5743810, and/or rs4129009 is used as a biomarker to predict or determine whether a patient is likely to respond or benefit from treatment with a STING agonist (e.g., E7766 or a pharmaceutically acceptable salt thereof). In some embodiments, the patient carries: (i) reference or heterozygous DNA sequences for TMEM173 V48V (rs7447927); and (ii) reference or heterozygous DNA sequences for TLR6 S249P (rs5743810); or reference DNA sequences for TLR10 I775V (rs4129009). In some embodiments, the patient is reference for rs7447927 and reference for rs5743810. In some embodiments, the patient is reference for rs7447927 and heterozygous for rs5743810. In some embodiments, the patient is heterozygous for rs7447927 and reference for rs5743810. In some embodiments, the patient is heterozygous for rs7447927 and heterozygous for rs5743810. In some embodiments, the patient is reference for rs7447927 and reference for rs4129009. In some embodiments, the patient is heterozygous for rs7447927 and reference for rs4129009. In some embodiments, the patient's genotype for single nucleotide variant(s) rs7447927, rs5743810, and/or rs4129009 is determined by DNA variant testing.

In some embodiments, the STING agonist is administered to the patient intratumorally. In some embodiments, the STING agonist is administered to the patient subcutaneously. In some embodiments, the STING agonist is administered to the patient via an intra-tumoral injection. In some embodiments, the injection is administered into a solid tumor, e.g., wherein the tumor has a long-axis diameter of at least 1 centimeter. In some embodiments, the injection is administered into a lymph node, e.g., wherein the lymph node has a short-axis diameter of at least 1.5 centimeters. In some embodiments, the STING agonist is administered systemically. In some embodiments, the STING agonist is administered to the patient via an intravenous infusion. In some embodiments, a person of skill in the art could select suitable concentrations and dosing frequencies of the STING agonist to be administered to the patient.

In some embodiments, the STING agonist is administered to the patient in combination with at least one additional therapy. In some embodiments, the delivery of one treatment is still occurring when the delivery of the second treatment begins, so that there is overlap. In some embodiments, the first and second treatment are initiated at the same time. These types of delivery are sometimes referred to herein as “simultaneous,” “concurrent,” or “concomitant” delivery. In other embodiments, the delivery of at least one dose of one treatment ends before delivery of a dose of the second treatment begins. This type of delivery is sometimes referred to herein as “successive” or “sequential” delivery. In some embodiments, a STING agonist is administered to a patient alone or in combination with at least one additional therapy. In some embodiments of combination treatment, the at least one additional therapy comprises administering a checkpoint inhibitor. In some embodiments, a STING agonist and a checkpoint inhibitor are administered simultaneously. In some embodiments, a STING agonist and a checkpoint inhibitor are administered sequentially. In either case, the two treatments should be administered sufficiently close in time so as to provide the desired therapeutic effect.

In some embodiments, the at least one additional therapy comprises administering one or more checkpoint inhibitors. In some embodiments, the checkpoint inhibitor targets PD1, PDL1, and/or CTLA4. In some embodiments, the checkpoint inhibitor comprises a programmed death-1 pathway (PD1) inhibitor. In some embodiments, the PD1 inhibitor comprises an anti-PD1 antibody. In some embodiments, the anti-PD1 antibody comprises pembrolizumab, nivolumab, and/or spartalizumab. In some embodiments, the PD1 inhibitor comprises an anti-PDL1 antibody. In some embodiments, the checkpoint inhibitor comprises a cytotoxic T-lymphocyte-associated antigen 4 pathway (CTLA4) inhibitor. In some embodiments, the CTLA4 inhibitor comprises an anti-CTLA4 antibody. In some embodiments, the anti-CTLA4 antibody comprises ipilimumab.

In some embodiments, the checkpoint inhibitor is an anti-PD1 antibody that binds to the PD-1 receptor. Exemplary anti-PD1 antibodies include but are not limited to pembrolizumab (MK-3475), nivolumab, and/or spartalizumab. Pembrolizumab and spartalizumab, for example, are both humanized monoclonal antibodies of the IgG4/kappa isotype designed to block the interaction between PD1 and its ligands, PDL1 and PDL2 (Kao and Lou (2019) Head Neck 41 Suppl 1:4-18; Naing et al. (2020) J Immunother Cancer 8 (1): e000530). Pembrolizumab enhances T lymphocyte immune responses in cultured blood cells from healthy human donors, cancer patients, and primates. Pembrolizumab has also been reported to modulate the level of interleukin-2 (IL-2), tumor necrosis factor alpha (TNFα), interferon gamma (IFNγ), and other cytokines. Nivolumab, for example, is a fully human IgG4 anti-PD1 monoclonal antibody that disrupts the interaction of the PD1 receptor with its ligands PDL1 and PDL2, thereby inhibiting the cellular immune response (Guo et al. (2017) J Cancer 8 (3): 410-6). In some embodiments, the anti-PD1 antibody is pembrolizumab. In some embodiments, the anti-PD1 antibody is spartalizumab. In some embodiments, the anti-PD1 antibody is nivolumab.

In some embodiments, the checkpoint inhibitor is an anti-PDL1 antibody. Exemplary anti-PDL1 antibodies include but are not limited to atezolizumab, avelumab, and durvalumab. Atezolizumab, for example, is an IgG1 humanized monoclonal antibody that is reported to block the PD1/PDL1 interaction, by targeting the expressed PDL1 on numerous kinds of malignant cells. This blockage of the PD1/PDL1 pathway may stimulate the immune defense mechanisms against tumors (Abdin et al. (2018) Cancers (Basel) 10 (2): 32). In some embodiments, the anti-PDL1 antibody is atezolizumab.

In other embodiments, a checkpoint inhibitor is an inhibitor of the cytotoxic T-lymphocyte-associated antigen (CTLA4) pathway. CTLA4, also known as CD152, is a protein receptor that downregulates immune responses. CTLA4 is constitutively expressed in regulatory T-cells, but only upregulated in conventional T-cells after activation.

As used herein, the term “CTLA4 inhibitor” refers to any inhibitor of CTLA4 and/or the CTLA4 pathway unless the context indicates that it refers specifically to an inhibitor that acts directly on CTLA4. Exemplary CTLA4 inhibitors include but are not limited to anti-CTLA4 antibodies. In some embodiments, the CTLA4 inhibitor comprises an anti-CTLA4 antibody. Exemplary anti-CTLA4 antibodies include but are not limited to ipilimumab (MDX-010) and tremelimumab (CP-675,206), both of which are fully human. Ipilimumab is an IgG1 with a plasma half-life of approximately 12-14 days; tremelimumab is an IgG2 with a plasma half-life of approximately 22 days. See, e.g., Phan et al. (2003) Proc Natl Acad Sci USA. 100:8372-7; Ribas et al. (2005) J Clin Oncol. 23:8968-77; Weber et al. (2008) J Clin Oncol. 26:5950-6. In some embodiments, the anti-CTLA4 antibody comprises ipilimumab.

In some embodiments, a checkpoint inhibitor is targeted at PD1/PDL1, CTLA4, OX40, CD40, LAG3, TIM3, GITR, and/or KIR (including a multispecific antibody that may bind to more than one epitope on one or more checkpoint antigen targets). In some embodiments, a checkpoint inhibitor is targeted at CTLA4, OX40, CD40, and/or GITR. In some embodiments, a checkpoint inhibitor is an inhibitory antibody or other similar inhibitory molecule (e.g., an inhibitory anti-CTLA4 or anti-PD1/PDL1 antibody). In some embodiments, a checkpoint inhibitor is an agonist for the target; examples of targets in this class include the stimulatory targets OX40, CD40, and/or GITR. In some embodiments, the checkpoint inhibitor is an agonist antibody for OX40, CD40, and/or GITR. Agonist antibodies directed against OX40 may have a dual role, inhibiting regulatory T-cell suppression, while enhancing effector T-cell functions. Agonist anti-GITR antibodies have also been shown to make effector T-cells more resistant to the inhibition induced by regulatory T-cells (Karaki et al. (2016) Vaccines (Basel) 4 (4): 37). Likewise, agonist CD40 antibodies may demonstrate T-cell-dependent anti-tumor activity. Activation of CD40 on dendritic cells may increase cross-presentation of tumor antigens and consequently the number of activated tumor-directed effector T-cells (Ellmark et al. (2015) Oncoimmunol. 4 (7): e1011484).

In some embodiments, the STING agonist is administered to the patient in combination with at least one additional therapy. In some embodiments, the additional therapy is administered to the subject before administration of the STING agonist. In some embodiments, the additional therapy is administered to the subject simultaneously with the administration of the STING agonist. In some embodiments, the additional therapy is administered to the subject following administration of the STING agonist. In some embodiments, the additional therapy is administered to the patient intratumorally or intravascularly. In some embodiments, the additional therapy is administered to the patient via an intra-tumoral injection. In some embodiments, the injection is administered into a solid tumor, e.g., wherein the tumor has a long-axis diameter of at least 1 centimeter. In some embodiments, the injection is administered into a lymph node, e.g., wherein the lymph node has a short-axis diameter of at least 1.5 centimeters. In some embodiments, the additional therapy is administered systemically. In some embodiments, the additional therapy is administered to the patient via an intravenous infusion. In some embodiments, a person of skill in the art could select suitable routes of administration, concentrations, and dosing frequencies of the additional therapeutic to be administered to the patient.

In some embodiments, a biological response is evaluated in a sample after contacting with one or more agents, e.g., a STING agonist (e.g., E7766 or a pharmaceutically acceptable salt thereof), measured using any of the exemplary assays described herein or known in the art. In some embodiments, the assay involves contacting a subject (e.g., patient), cell, or culture of cells with a STING agonist (e.g., E7766 or a pharmaceutically acceptable salt thereof) and determining whether one or more properties of the subject, cell, or culture changes after contact. In some embodiments, a change may be detected in a level of RNA expression, a level of protein expression, a level of protein activity, a level of protein modification (e.g., protein phosphorylation), a level of one or more cellular function(s), a level of a reporter signal, toxicity, cytokine release, cell proliferation, cell motility or morphology, cell growth, cell death (e.g., apoptosis), and/or tumor growth.

In some embodiments, the biological response is detected using one or more assays to evaluate protein expression, activity, and/or phosphorylation level. In some embodiments, the biological response is detected using one or more assays selected from a kinase or enzymatic activity assay, incubation of whole cells with radiolabeled ³²P-orthophosphate, two-dimensional gel electrophoresis, an immunoblot assay (e.g., Western blot), an AlphaLISA® assay, an enzyme-linked immunosorbent assay (ELISA), a cell-based ELISA assay, intracellular flow cytometry, immunocytochemistry (ICC), immunohistochemistry (IHC), mass spectrometry, multi-analyte profiling (e.g., a phospho-protein multiplex assay), and fluorescent in situ hybridization (FISH). In some embodiments, the biological response is detected by measuring or monitoring tumor growth over a period of time.

In some embodiments, the biological response is an increase or decrease in the expression or activity of at least one protein in the patient, as compared to the expression or activity of the same protein(s) in the patient prior to administration of the STING agonist. In some embodiments, the biological response is an increase or decrease in the expression or activity of at least one protein in a sample from the patient, as compared to the expression or activity of the same protein(s) in the sample prior to contact with the STING agonist. In some embodiments, the at least one protein having increased or decreased expression or activity comprises a cytokine and/or a chemokine.

In some embodiments, the biological response is detected using imaging of the patient. In some embodiments, the biological response is detected by measuring or monitoring tumor growth over a period of time. In some embodiments, the biological response is detected by measuring or monitoring using CT/MRI and/or photography of the patient. In some embodiments, treatment is continued if a biological response is detected.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1. Intratumorally Administered STING Agonist Compound 1 in Patients with Advanced Solid Tumors or Lymphomas and Dose Extension Cohort

A phase 1/1b, open label, multicenter study is performed to assess safety/tolerability and preliminary clinical activity of Compound 1 as a single agent administered intratumorally in patients with advanced solid tumors or lymphomas. In the Dose Escalation and Dose Expansion Part, patients will receive Compound 1 intratumorally in advanced solid tumors or lymphomas to assess safety/tolerability profile of Compound 1 and to determine the maximum tolerated dose (MTD) and/or recommended Phase 2 dose (RP2D). Toxicity will be evaluated according to National Cancer Institute Common Terminology Criteria for Adverse Events version 5.0 (NCI CTCAE v.5.0). Patients will also be evaluated for the incidence of Adverse Events (AEs) and Serious Adverse Events (SAEs) for up to 90 days after the last dose of Compound 1.

In the Dose Expansion Part, patients with melanoma, head and neck squamous cell carcinoma (HNSCC), breast cancer, colorectal cancer, and/or other tumors including lymphomas will receive Compound 1 intratumorally to confirm safety and assess preliminary clinical activity of Compound 1 as a single agent. Clinical activity will be evaluated by objective response rate (ORR), duration of response (DOR), and disease control rate (DCR) on treatment with Compound 1. Patients will also be evaluated for the incidence of Adverse Events (AEs) and Serious Adverse Events (SAEs) for up to 90 days after the last dose of Compound 1.

### Example 2. Biomarkers for STING Agonist Treatment

Background: Admixture of archaic (Neandertal and Denisova) and ancestral genes may modulate susceptibility to autoimmunity and cancer. Functional interactions between archaic and ancestral STING1 and TLR genes were investigated to identify vulnerabilities that may be addressed by STING agonist therapy.

Methods: Gene variants from 10,389 cancer patients were obtained from TCGA. Archaic sequences were accessed using UCSD genome browser v410. Linkage disequilibrium was investigated using LDlink v5.0. Patient 1 was treated according to the procedures discussed in Example 1. See also ClinicalTrials.gov Identifier: NCT04144140, which is incorporated herein by reference.

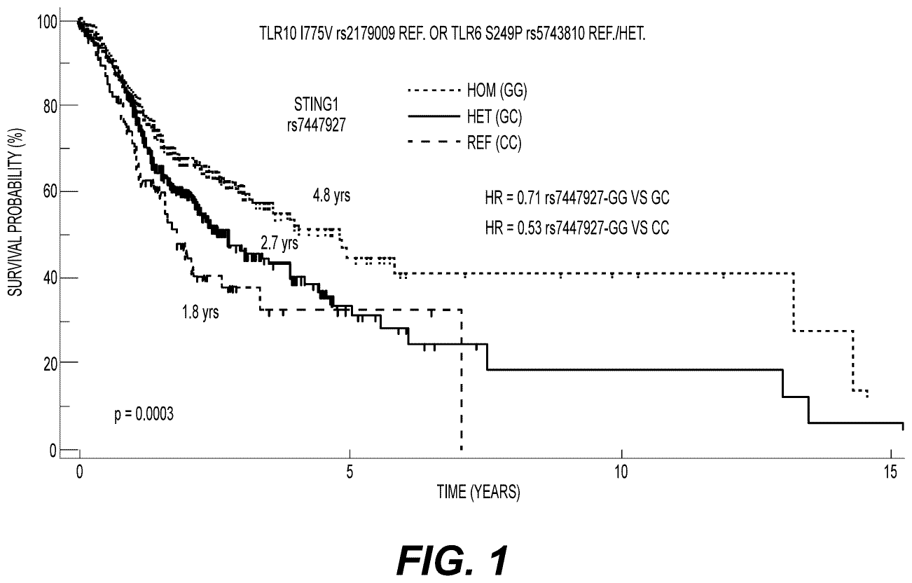

Results: STING1 variants were overrepresented in cancer patients. The common V48V (rs7447927-C>G) variant was in linkage disequilibrium with the reference alleles of the partially active HAQ and REF variants, and with rs13153461, present in Neandertal sequences. STING1 rs7447927-G was also associated with HLA A*24:02 (p<0.001), A*02:06 (p=0.01), and A*31:01 (p=0.02), of Neandertal origin. The potential for epistasis between STING1 rs7447927-G and TLR variants associated with decreased H. pylori prevalence (e.g., Neandertal-associated TLR10 I775V (rs4129009) and ancestral TLR6 P249S (rs5743810)) was also investigated. No independent prognosis was identified in 32 TCGA legacy studies; however, in upper aerodigestive tract (oral cavity, oropharynx, esophageal, gastric, biliary tract) tumor patients who carried reference/heterozygous TLR6 S249 and/or reference TLR10 I775, in combination with Neandertal-associated STING1 rs7447927-G zygosity was a predictor of survival. Hazards ratio for rs7447927-GG vs GC (4.8 vs 2.7 yrs. median survival), and GG vs CC (4.8 vs 1.8 yrs.) were 0.71 and 0.53 (N=713, p=0.0003), respectively (FIG. 1).

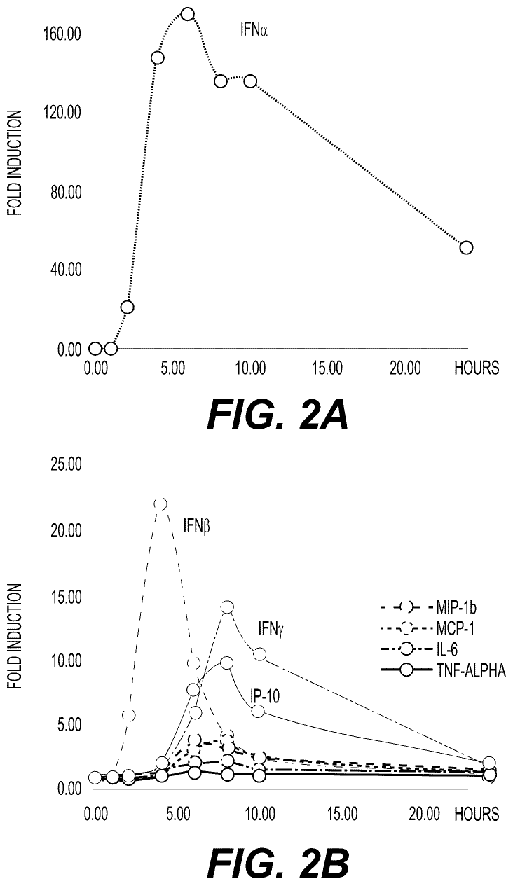

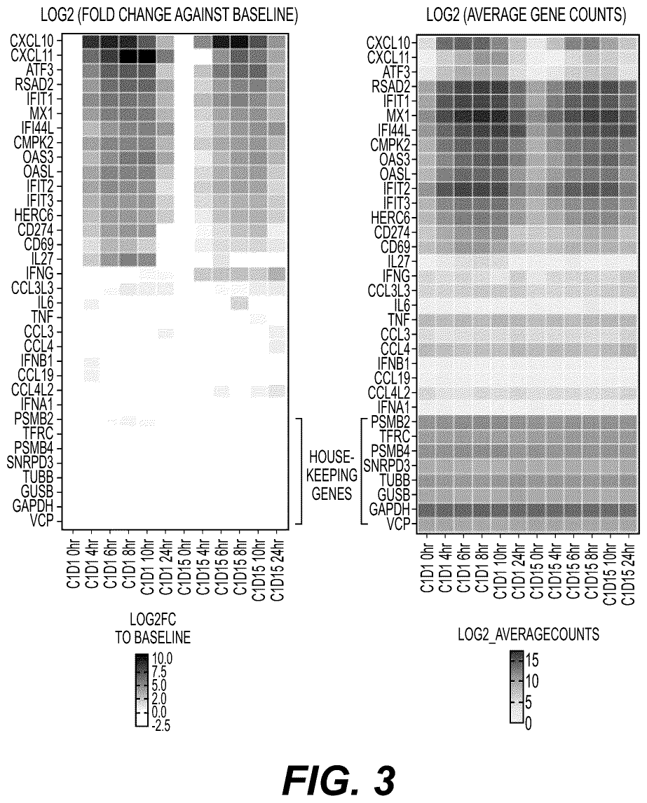

An initial case study of a first patient receiving a STING agonist (Patient 1) was evaluated. Patient 1, a 75 year-old esophageal cancer patient with a history of gastroesophageal reflux disease (GERD), Barrett's esophagus, and TLR6 S249, TLR10 I775V and STING1 rs7447927-GC, was progressing from anti-PD1 and chemotherapy and received 11 intra-tumoral injections of 75 μg of the STING agonist Compound 1. Patient 1 received the first injection on day 1, followed by 3 weekly administrations, and 7 administrations on 3-week intervals. Non treatment-related grade 2 anemia and hyponatremia, serum IFN-β and IP10 induction, 20% tumor size reduction including abscopal effects, and 6.3 months progression-free survival (PFS) benefit were observed (FIGS. 2 and 3).

1. A method of treating a cancer in a patient, comprising administering a therapeutically effective amount of a STING agonist to the patient who carries:
(i) reference or heterozygous DNA sequences for TMEM173 V48V (rs7447927); and
(ii) reference or heterozygous DNA sequences for TLR6 S249P (rs5743810); or reference DNA sequences for TLR10 I775V (rs4129009).

2. A method of treating a cancer in a patient, comprising:
(a) determining that the patient carries:
(i) reference or heterozygous DNA sequences for TMEM173 V48V (rs7447927); and
(ii) reference or heterozygous DNA sequences for TLR6 S249P (rs5743810); or reference DNA sequences for TLR10 I775V (rs4129009); and
(b) administering a therapeutically effective amount of a STING agonist to the patient.

3. A method of identifying a cancer patient suitable for treatment with a STING agonist, comprising:
(a) determining that the patient carries:
(i) reference or heterozygous DNA sequences for TMEM173 V48V (rs7447927); and
(ii) reference or heterozygous DNA sequences for TLR6 S249P (rs5743810); or reference DNA sequences for TLR10 I775V (rs4129009); and
(b) identifying the patient as suitable for treatment with a STING agonist.

4. The method of any one of claims 1 to 3, further comprising obtaining a biological sample from the patient, and determining from the sample the patient's genotype for single nucleotide variant(s) rs7447927, rs5743810, and/or rs4129009.

5. The method of claim 4, wherein the biological sample comprises a buccal sample, a blood sample, and/or a tumor sample.

6. The method of claim 4 or claim 5, wherein the biological sample is obtained by a buccal swab, a phlebotomy, or a tumor biopsy.

7. The method of any one of claims 1 to 6, wherein the patient's genotype for single nucleotide variant(s) rs7447927, rs5743810, and/or rs4129009 is determined by DNA variant testing.

8. The method of claim 7, wherein the DNA variant testing comprises next generation sequencing (NGS), polymerase chain reaction (PCR), or pyrosequencing.

9. The method of any one of claims 1 to 8, wherein the patient is reference for rs7447927 and reference for rs5743810.

10. The method of any one of claims 1 to 8, wherein the patient is reference for rs7447927 and heterozygous for rs5743810.

11. The method of any one of claims 1 to 8, wherein the patient is heterozygous for rs7447927 and reference for rs5743810.

12. The method of any one of claims 1 to 8, wherein the patient is heterozygous for rs7447927 and heterozygous for rs5743810.

13. The method of any one of claims 1 to 8, wherein the patient is reference for rs7447927 and reference for rs4129009.

14. The method of any one of claims 1 to 8, wherein the patient is heterozygous for rs7447927 and reference for rs4129009.

15. The method of any one of claims 1 to 14, wherein the cancer comprises a cancer of the upper aerodigestive tract.

16. The method of any one of claims 1 to 15, wherein the cancer comprises a cancer of the lip, buccal mucosa, floor of mouth, oral cavity, hard palate, base of tongue, oral tongue, tonsil, oropharynx, salivary gland, gallbladder, esophagus, stomach, and/or biliary tract.

17. The method of any one of claims 1 to 16, wherein the cancer comprises a head and neck squamous cell carcinoma (HNSCC).

18. The method of any one of claims 1 to 16, wherein the cancer comprises an esophageal cancer.

19. The method of any one of claims 1 to 18, wherein the cancer is a recurrent cancer.

20. The method of any one of claims 1 to 19, wherein the cancer is a metastatic cancer.

21. The method of any one of claims 1 to 20, wherein the STING agonist comprises one or more of E7766, ADU-S100, BI 1387446, MK-1454, MK-2118, BMS-986301, CDK-002, GSK-3745417, idronoxil, SB-11285, IMSA-101, SNX-281, SYNB-1891, TAK-676, DMXAA, FAA, CMA, α-Mangostin, BNBC, DSDP, diABZI, a bicyclic benzamide, a benzothiophene, MSA-2, SR-717, MAVU-104, TTI-10001, SRCB-0001, CRD 5500, ALG-031048, JNJ-'6196, IACS-8803, IACS-8779, NZ-IO-STING, OX-401, SITX-799, STACT-TREX1, XMT-2056, ONM-500, MV-626, PF-06928215, DN-15089, HH18202, STI-001, and pharmaceutically acceptable salts thereof.

22. The method of any one of claims 1 to 21, wherein the STING agonist comprises one or more of E7766, ADU-S100, BI 1387446, MK-1454, MK-2118, BMS-986301, CDK-002, GSK-3745417, idronoxil, SB-11285, IMSA-101, SNX-281, SYNB-1891, TAK-676, and pharmaceutically acceptable salts thereof.

23. The method of any one of claims 1 to 22, wherein the STING agonist comprises a diammonium salt of E7766.

24. The method of any one of claims 1 to 23, wherein the STING agonist is administered to the patient intratumorally.

25. The method of any one of claims 1 to 24, wherein the STING agonist is administered to the patient via an intra-tumoral injection.

26. The method of claim 25, wherein the injection is administered into a solid tumor, e.g., wherein the tumor has a long-axis diameter of at least 1 centimeter.

27. The method of claim 25, wherein the injection is administered into a lymph node, e.g., wherein the lymph node has a short-axis diameter of at least 1.5 centimeters.

28. The method of any one of claims 1 to 27, wherein the STING agonist is administered to the patient in combination with at least one additional therapy.

29. The method of claim 28, wherein the at least one additional therapy comprises administering a checkpoint inhibitor.

30. The method of claim 29, wherein the checkpoint inhibitor targets PD1, PDL1, and/or CTLA4.

31. The method of claim 29 or claim 30, wherein the checkpoint inhibitor comprises a programmed death-1 pathway (PD1) inhibitor.

32. The method of claim 31, wherein the PD1 inhibitor comprises an anti-PD1 antibody.

33. The method of claim 32, wherein the anti-PD1 antibody comprises pembrolizumab, nivolumab, and/or spartalizumab.

34. The method of claim 31, wherein the PD1 inhibitor comprises an anti-PDL1 antibody.

35. The method of any one of claims 29 to 30, wherein the checkpoint inhibitor comprises a cytotoxic T-lymphocyte-associated antigen 4 pathway (CTLA4) inhibitor.

36. The method of claim 35, wherein the CTLA4 inhibitor comprises an anti-CTLA4 antibody.

37. The method of claim 36, wherein the anti-CTLA4 antibody comprises ipilimumab.

38. The method of any one of claims 1 to 37, wherein treatment reduces or inhibits growth of a tumor in the patient, as compared to the growth of the tumor prior to treatment.

39. The method of any one of claims 1 to 38, wherein treatment reduces the size of a tumor in the patient, as compared to the size of the tumor prior to treatment.

40. The method of any one of claims 1 to 39, wherein treatment reduces the size of a tumor in the patient by at least 5%, 10%, 15%, 20%, or more, as compared to the size of the tumor prior to treatment.

41. The method of any one of claims 1 to 40, wherein treatment reduces the size of a tumor in the patient by at least 20% or more, as compared to the size of the tumor prior to treatment.

42. The method of any one of claims 1 to 41, wherein treatment increases or decreases the expression or activity of at least one protein in the patient, as compared to the expression or activity of the same protein(s) prior to treatment.

43. The method of claim 42, wherein the at least one protein comprises a cytokine and/or a chemokine.

44. The method of claim 42 or claim 43, wherein the at least one protein comprises IFNα, IFNβ, IFNγ, IP-10, MCP-1, MIP-1b, IL-6, and/or TNFα.

45. A STING agonist for use in treating a cancer in a patient, comprising administering a therapeutically effective amount of the STING agonist to the patient who carries:
(i) reference or heterozygous DNA sequences for TMEM173 V48V (rs7447927); and
(ii) reference or heterozygous DNA sequences for TLR6 S249P (rs5743810); or reference DNA sequences for TLR10 I775V (rs4129009).
